(19) United States
(12) Patent Application Publication
(10) Pub. No.: US 2025/0346723 A1
(43) Pub. Date: Nov. 13, 2025

(54) TELLURIUM-CONTAINING POLYMER AND COMPOUND

(71) Applicants: The School Corporation Kansai University, Suita-shi, Osaka (JP); Mitsubishi Gas Chemical Company, Inc., Chiyoda-ku, Tokyo (JP)

(72) Inventors: Hiroto KUDO, Suita-shi, Osaka (JP); Tadashi OMATSU, Hiratsuka-shi, Kanagawa (JP); Takashi SATO, Hiratsuka-shi, Kanagawa (JP); Masatoshi ECHIGO, Chiyoda-ku, Tokyo (JP)

(21) Appl. No.: 18/862,395

(22) PCT Filed: May 2, 2023
(86) PCT No.: PCT/JP2023/017211

§ 371 Date: Nov. 1, 2024

(30) Foreign Application Priority Data
May 2, 2022 (JP) 2022-076308

(51) Int. Cl. C08G 79/00; C07C 395/00; G03F 7/038; G03F 7/11

(52) U.S. Cl. CPC C08G 79/00; C07C 395/00; G03F 7/038; G03F 7/11

(57) ABSTRACT

A polymer including tellurium in the main chain, which is prepared by reacting (A) a compound having a plurality of cyclic (thio) ether groups, and (B) TeX₄, wherein X is a halogen atom.

### TECHNICAL FIELD

The present invention relates to a tellurium-containing polymer and compound.

### BACKGROUND ART

Tellurium (Te) has properties such as electronegativity, ionization energy or electron affinity comparable to oxygen, sulfur and selenium. Patent document 1 discloses that Te-containing compounds and resins are useful as resist materials and optical articles.

### CITATION LIST

### Patent Literature

PTL 1: International Publication No. WO2020/226150

### SUMMARY OF INVENTION

### Technical Problem

While the compounds and others disclosed in Patent Document 1 are industrially useful, the inventors were inspired by the idea that if another type of Te-containing polymer could be provided, the range of industrially useful material options may be expanded. In particular, compounds containing Te and oxygen or sulfur atoms may increase the refractive index or further improve sensitivity when used as a resist material. In view of these circumstances, an object of the present invention is to provide a compound having an ether bond or thioether bond with Te.

### Solution to Problem

The inventors have solved the above problem by allowing a compound containing a cyclic (thio) ether group to react with tellurium tetrahalide. Accordingly, the above problem is solved by the following present invention.

Mode 1

A polymer including tellurium in the main chain, which is prepared by reacting:
(A) a compound having a plurality of cyclic (thio) ether groups, and
(B) TeX₄, wherein X is a halogen atom.

Mode 2

The polymer according to mode 1, wherein (A) is a compound represented by formula (A1) described later.

Mode 3

The polymer according to mode 2, wherein
W in the formula is a group represented by —Z—Ar—Z—, and
Ar is an organic group having an aromatic ring or a heterocyclic ring.

Mode 4

The polymer according to mode 3, wherein
Ar is a group represented by —Ar′—U—Ar′—
Ar′ is an aromatic group or a heterocyclic aromatic group, and
U is a divalent organic group or a single bond.

Mode 5

The polymer according to mode 1 or 2, wherein the cyclic (thio) ether group of (A) has a 3 to 5-membered ring structure.

Mode 6

A method for producing the polymer according to any of modes 1 to 4, including a step of subjecting (A) and (B) to an addition reaction.

Mode 7

The method according to mode 6, wherein the reaction is performed in the presence of Lewis acid.

Mode 8

A composition including the polymer according to any of modes 1 to 4.

Mode 9

The composition according to mode 8, including a component selected from the group consisting of a solvent, an acid generating agent, an acid crosslinking agent and a combination thereof.

Mode 10

A resist film formed from the composition according to mode 8.

Mode 11

A method for forming a pattern, including:
a film formation step of forming a film on a substrate by use of the composition according to mode 8,
an exposure step of exposing the film, and
a development step of developing the film exposed in the exposure step, to thereby form a pattern.

Mode 12

An underlayer film for lithography formed by use of the composition according to mode 8.

Mode 13

An optical component formed by use of the composition according to mode 8.

Mode 14

A tellurium-containing compound prepared by reacting
(a) a compound having one cyclic (thio) ether group, and
(B) TeX₄, wherein X is a halogen atom.

Mode 15

A composition including the tellurium-containing compound according to mode 14.

Mode 16

A purification method including:
a step of dissolving the tellurium-containing polymer according to any of modes 1 to 4 or the tellurium-containing compound according to mode 14 in a solvent including an organic solvent incompatible with water to thereby obtain a solution, and
a first extraction step of bringing the resulting solution into contact with an acidic aqueous solution to extract impurities in the polymer or compound.

### Advantageous Effects of Invention

The present invention can provide a polymer and a compound having an ether bond or thioether bond with Te.

### BRIEF DESCRIPTION OF DRAWINGS

FIG. 1 shows a ¹H-NMR spectrum and a ¹³C-NMR spectrum of BPAO obtained in Example 1.

FIG. 2 shows ¹H-NMR spectra of the reaction solution obtained in run 1 to run 5 in Table 1.

FIG. 3 shows a ¹H-NMR spectrum of the polymer after purification obtained in Example 1.

FIG. 4 shows a photograph of the film obtained in Example 2.

FIG. 5 shows a ¹H-NMR spectrum of the compound obtained in Example 3.

FIG. 6 shows a ¹H-NMR spectrum of tBPO obtained in Example 4.

FIG. 7 shows a ¹H-NMR spectrum of the polymer obtained in Example 7.

### DESCRIPTION OF EMBODIMENTS

In the following, the present invention will be described in detail. In the present invention, for “X to Y”, the end values are inclusive.

### 1. Polymer

The polymer according to the present embodiment is obtained by reacting (A) a compound having a plurality of cyclic (thio) ether groups, and (B) TeX₄, wherein X is a halogen atom.

### (1) (A) Compound Having a Plurality of Cyclic (Thio) Ether Groups

The compound having a plurality of cyclic (thio) ether groups (hereinafter may be referred to as “compound A”) has a plurality of cyclic ether groups or cyclic thioether groups. The structure of the cyclic ether group and the cyclic thioether group is not limited, and is preferably a 3 to 5-membered ring structure and more preferably 3 or 4-membered ring structure.

Compound A is preferably represented by the following formula.

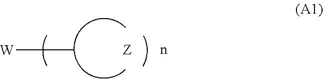

In the formula, Z is O or S, and the cyclic structure including Z is a group having a cyclic ether group or a cyclic thioether group, n represents the number of the groups, and is 2 to 4 and varies depending on the valence of W. n is preferably 2 or 3, more preferably 2 from the viewpoint of availability. The group having a cyclic ether group or a cyclic thioether group is preferably represented by the following formula (C1) or (C2). In the formula, R¹ is an alkylene group having 1 to 3 carbon atoms, R² is an alkyl group having 1 to 3 carbon atoms, and R⁴ is independently a hydrogen atom or an alkyl group having 1 or 2 carbon atoms. R⁴ is preferably a hydrogen atom from the viewpoint of reactivity.

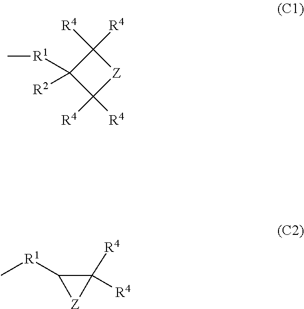

(C1) and (C2) are more preferably represented by the following formula (C1-1), (C2-1). In formula (C1), R² is preferably a methyl group or an ethyl group, and is more preferably a methyl group.

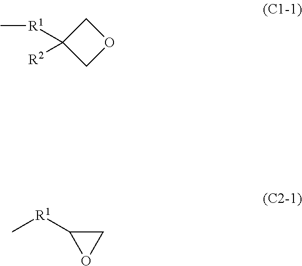

W is an organic group having 10 to 30 carbon atoms. The organic group is not limited, and is preferably a group having an aromatic group or a heterocyclic aromatic group. In an embodiment, W is a group represented by —Z—Ar—Z—. In other words, in this embodiment, n is 2 in formula (A1). Ar is an organic group having an aromatic ring and Z is O or S as described above. Ar may have a monocyclic structure or a polycyclic structure.

In a preferred embodiment, Ar is represented by —Ar′—U—Ar′—. Ar′ is an aromatic group or a heterocyclic aromatic group. In this case, compound A is represented by the following formula.

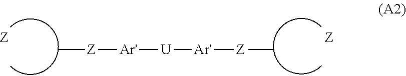

Ar′ is not limited, and is preferably a phenylene group, a naphthylene group, an anthracenediyl group, a phenanthrenediyl group, a pyrenediyl group or a fluorenediyl group. These groups may have a substituent. Unless otherwise defined, the “substituted” means that one or more hydrogen atoms in a functional group is/are each substituted with a substituent. Examples of the “substituent” include, but not particularly limited, a halogen atom, a hydroxy group, a cyano group, a nitro group, a thiol group, a heterocyclic group, a straight aliphatic hydrocarbon group having 1 to 20 carbon atoms, a branched aliphatic hydrocarbon group having 3 to 20 carbon atoms, a cyclic aliphatic hydrocarbon group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms or an alkylsilyl group having 1 to 20 carbon atoms.

U is a linking group, and more specifically a divalent organic group or a single bond. The organic group in this case is not limited, and is a heteroatom, or a divalent hydrocarbon group having 1 to 40 carbon atoms optionally having a heteroatom from the viewpoint of availability. The heteroatom refers to an atom other than carbon atom and a hydrogen atom; and examples thereof include an oxygen atom, a nitrogen atom, a sulfur atom, a fluorine atom, a chlorine atom, a bromine atom, an iodine atom and a silicon atom. A sulfur atom, an oxygen atom, a nitrogen atom and a silicon atom are preferred from the viewpoint of availability of the raw material. Examples of divalent hydrocarbon groups having 1 to 40 carbon atoms include a straight, branched or cyclic alkylene group having 1 to 40 carbon atoms and an aralkylene group having 6 to 40 carbon atoms. U is a single bond; a divalent heteroatom; a sulfone group; a straight, branched or cyclic alkylene group having 1 to 40 carbon atoms; an aralkylene group having 6 to 40 carbon atoms, and preferably a divalent alkylene having 1 to 5 carbon atoms, and particularly preferably a methylene group and an isopropylidene group from the viewpoint of easiness in synthesis. In particular, it is preferable that the —Ar′—U—Ar′— group is derived from bisphenol A, bisphenol F or bisphenol S. As described later, compound A may be a compound synthesized from bisphenol A and 3-(chloromethyl)-3-methyloxetane (BPAO). In that case-Ar′—U—Ar′— is derived from bisphenol A, and U is an isopropylidene group, Ar′ is a phenylene group and Z is an oxygen atom. The cyclic structure including Z is a group derived from 3-(chloromethyl)-3-methyloxetane.

In another embodiment, W is a group represented by Ar—(Z—)₃. In other words, in this embodiment, n is 3 in formula (A1). Ar is an organic group having an aromatic ring, Z is O or S as described above, and is bonded to the cyclic structure including Z. Ar may have a monocyclic structure or a polycyclic structure.

In a preferred embodiment, W is represented by U—(Ar′—Z—)₃. U is a trivalent linking group and preferably a trivalent linking group having 1 to 3 carbon atoms. Ar′ is an aromatic group or a heterocyclic aromatic group. In this case, compound A is represented by the following formula.

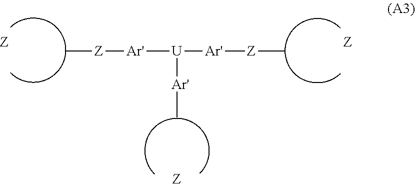

A preferred mode of linking group U in formula (A3) is a CH group, and is represented by the following formula (A4). Ar′ and Z in formula (A3) are as defined in (A2) above.

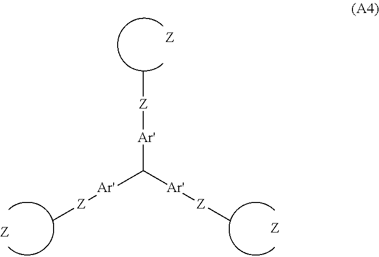

### (2) (B) TeX₄

TeX₄ (hereinafter also referred to as “Compound B”) is tellurium tetrahalide. X is halogen and preferably F, Cl or Br, and more preferably Cl.

### (3) Structure of Polymer

The polymer according to the present embodiment is obtained by addition reaction of compound A and compound B. By this reaction, Te and O or S derived from compound A are bonded to form a polymer. The estimated structure when compound A2 is used as compound A is shown below.

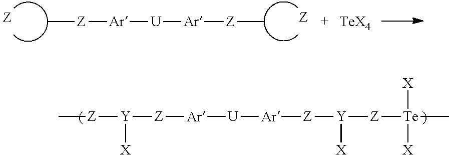

In the formula, Y is an alkylene group derived from a group having a cyclic (thio)ether group. This is addition reaction, and the resulting polymer has a bond of Te and O or S in the main chain. If the amount of compound B is excessive, an outstandingly high molecular weight component is formed and the molecular weight distribution becomes multimodal. This is presumably due to the ring-opening polymerization of compound A alone, since compound B has Lewis acid properties. However, if compound A and B are added in a near equimolar molar ratio, ring-opening polymerization of compound A alone does not occur. From the above, the polymer is formed by addition reaction of compound A and B, and is presumed to have the above structure.

For example, the estimated structure of a polymer synthesized using a compound with a bisphenol A backbone and two oxetane rings (hereinafter also referred to as “BPAO”) as compound A and TeCl₄ as compound B is as follows.

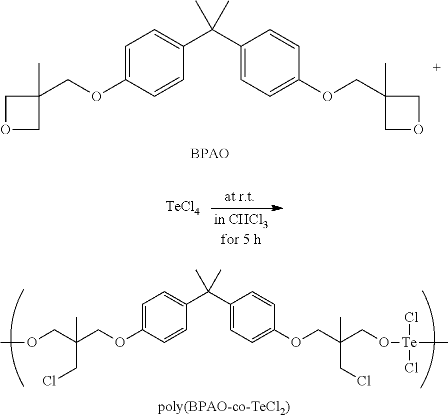

When compound A is a trifunctional compound as represented by formula (A3), the resulting polymer is a hyperbranched polymer with a hyperbranched structure. Hyperbranched polymers have branched chains extending from the main chain, but the branched chains are not long enough to form a gel. For example, the hyperbranched polymer has the following estimated structure.

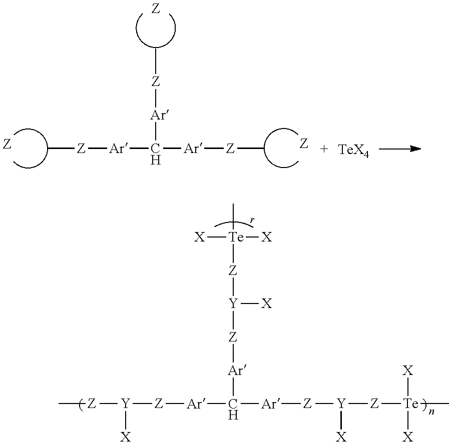

n is the repeating number in the main chain, r is the repeating number in the branched chain, and n>r. An excessively large r causes gelation and an excessively small r reduces solubility in solvent. In an embodiment, r may be about 10 to 60% of n.

Below is the estimated structure of a hyperbranched polymer obtained from MTPOX and TeCl₄ as an example when compound A is a trifunctional compound as represented by formula (A3).

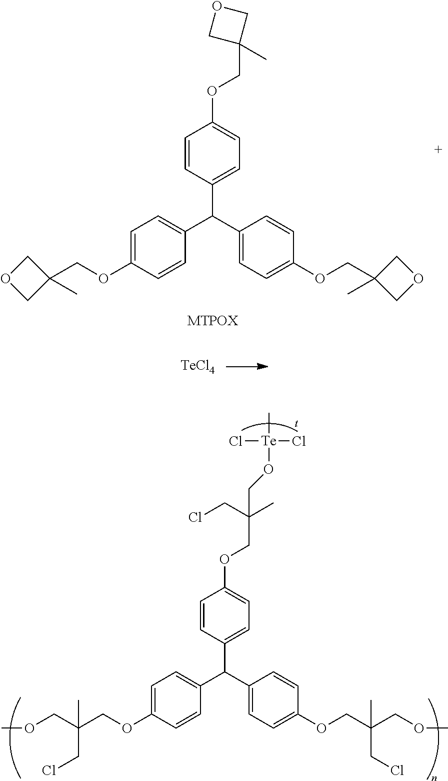

### (4) Properties

The polymer contains Te. Te easily absorbs UV light. Thus, the polymer is useful as a resist material. Furthermore, the polymer is transparent, and as the polymer includes a Te—O or Te—S bond, it has a high refractive index. Therefore, the polymer is also useful as an optical component.

### 2. Compound

The compound according to the present embodiment is obtained by reacting (a) a compound having one cyclic (thio) ether group (hereinafter also referred to as “compound a”) and compound B. Compound a is as described for compound A except for including one cyclic (thio) ether group. For example, compound a is preferably represented by the following formula.

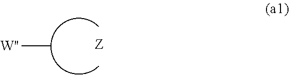

In the formula, Z is O or S, and the cyclic structure including Z is as defined for compound A.

W″ is an organic group having 1 to 30 carbon atoms or a halogen atom. The organic group is not limited, and is preferably an alkyl group, or a group having an aromatic group or a heterocyclic aromatic group. The alkyl group here is preferably a straight, branched or cyclic alkyl group having 1 to 30 carbon atoms. In an embodiment, W is a group having an aromatic group or a heterocyclic aromatic group, and is preferably a group represented by —Z—Ar”. Ar“ is an aromatic group or a heterocyclic aromatic group, and may have a monocyclic structure or a polycyclic structure. In a preferred embodiment, Ar″ is a phenyl group, a naphthyl group, an anthracenyl group, a phenanthrenyl group, a pyrenyl group or a fluorenyl group. These groups may have a substituent. Unless otherwise defined, the “substituted” means that one or more hydrogen atoms in a functional group is/are each substituted with a substituent. Examples of the “substituent” include, but are not limited to, preferably a halogen atom, a hydroxy group, a cyano group, a nitro group, a thiol group, a heterocyclic group, a straight alkyl group having 1 to 20 carbon atoms, a branched alkyl group having 3 to 20 carbon atoms, a cyclic alkyl group having 3 to 20 carbon atoms, an aryl group having 6 to 20 carbon atoms, an alkoxyl group having 1 to 20 carbon atoms, an amino group having 0 to 20 carbon atoms, an alkenyl group having 2 to 20 carbon atoms, an alkynyl group having 2 to 20 carbon atoms, an acyl group having 1 to 20 carbon atoms, an alkoxycarbonyl group having 2 to 20 carbon atoms, an alkyloyloxy group having 1 to 20 carbon atoms, an aryloyloxy group having 7 to 30 carbon atoms or an alkylsilyl group having 1 to 20 carbon atoms. The substituent is particularly preferably a straight or branched alkyl group having 1 to 6 carbon atoms.

For example, the scheme and the estimated structure of a compound synthesized using 3-(chloromethyl)-3-methyloxetane as compound a and TeCl₄ as compound B are as follows.

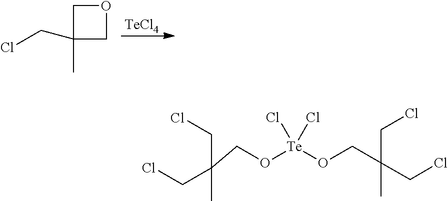

Halogen bonded to the alkyl group of the present compound (Cl in the illustrated compound) may be converted to another functional group as shown in the following scheme. For example, a compound in which halogen is converted to a polymerizable functional group is useful for a polymer containing Te. R is an organic group in the following scheme.

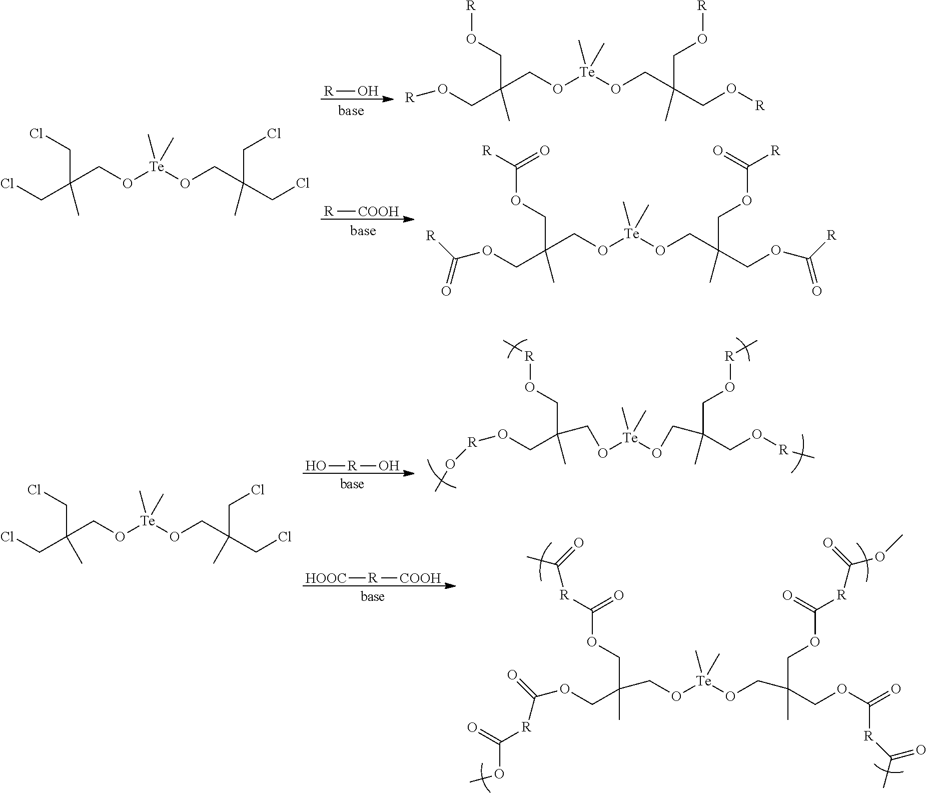

Furthermore, the scheme and the estimated structure of a compound synthesized using 3-(p-tBu-phenoxy)-3-methyloxetane as compound a and TeCl₄ as compound B are as follows.

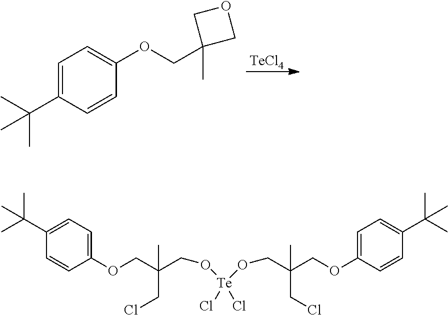

Halogen bonded to the alkyl group of the present compound (Cl in the illustrated compound) may be converted to another functional group as described above. 3. A method for producing polymer

The polymer compound according to the present embodiment may be produced by addition reaction of compound A and compound B. Reaction conditions are not limited, and in an embodiment, the temperature is in the range of −70° C. to 200° C., preferably 0° C. to 80° C., more preferably 20 to 50° C., and further preferably room temperature (20° C.). For the atmosphere, it is preferable to carry out the reaction at normal pressure under nitrogen, or the reaction may also be performed in vacuum. A solvent may also be used in the reaction. The solvent is not limited, and halogen hydrocarbon such as chloroform, methylene chloride and chlorobenzene, an ether compound such as tetrahydrofuran and dioxane, an aromatic compound such as benzene, toluene and nitrobenzene, and an aprotic polar compound such as dimethyl sulfoxide, 1-methyl-2-pyrrolidone, dimethylformamide and dimethylacetamide may be used. Te in compound B reacts with two cyclic (thio) ether groups. Thus, it is preferable to determine the number of moles of the two so that the equivalent of cyclic (thio) ether in compound A and the equivalent of Te in compound B is about 1:1. The equivalent of cyclic (thio) ether and the equivalent of Te may be about 1:(0.7 to 1.2). It is preferable to use Lewis acid such as aluminum chloride, tin chloride and boron trichloride as the catalyst. The amount is not limited, and is about 0.01 to 0.1 equivalent relative to compound A. However, boron trifluoride diethyl ether may promote the self-polymerization of compound A, and thus its amount should be adjusted accordingly.

### 4. Method for Producing Compound

The polymer compound according to the present embodiment may be produced by addition reaction of compound A or compound a and compound B. Reaction conditions and others are as described for the method for producing polymer.

### 5. Composition

### [Composition]

A compound including the polymer synthesized from compound A and compound B above, the compound synthesized from compound A or compound a and compound B above, or a polymer derived from the compound is useful as a material for lithography, a material composition for lithography, a composition for resist underlayer film formation and a composition for optical article formation.

### [Material for Lithography]

The material for lithography is a material usable in lithography techniques, and may be used as a material composition for lithography, and may also be used in, for example, a resist application (namely, resist composition), an application for forming an intermediate layer (namely, a composition for intermediate layer formation) and an application for forming an underlayer film (namely, a composition for underlayer film formation).

The material for lithography may be used, for example, for a resist composition which can reduce film defects (achieve thin film formation), has good storage stability, is highly sensitive, has high refractive index and is transparent in specific wavelength ranges and can provide a favorable resist pattern shape. Since the material for lithography contains Te, the material is expected to be effective in sensitization, in particular, in lithography by EUV. Te is the second most effective sensitizer after xenon among all elements. The material for lithography can include no solvent.

### [Material Composition for Lithography]

A material composition for lithography includes the material for lithography according to the present embodiment, and a solvent. The material composition for lithography can reduce film defects (achieve thin film formation), can provide a favorable resist pattern shape and can form a film having high etching resistance. The material composition for lithography also has good storage stability and can form a resist pattern that is highly sensitive, and has high refractive index and is transparent in specific wavelength ranges.

### (Physical Properties and the Like of Material Composition for Lithography)

The material for lithography can be used in a resist application, as described above, and can form an amorphous film by a known method such as spin coating. A positive resist pattern and a negative resist pattern can be separately formed depending on the type of a developer used. Hereinafter, a case will be described where the material composition for lithography including the material for lithography of the present embodiment is used in a resist application (as a resist composition).

In a case where the material composition for lithography corresponds to a positive resist pattern, the rate of dissolution at 23° C. of an amorphous film formed by spin coating with the material composition for lithography, in a developer, is preferably 5 Å/sec or less, more preferably 0.0005 to 5 Å/sec, and further preferably 0.05 to 5 Å/sec. When the rate of dissolution is 5 Å/sec or less, a resist insoluble in the developer can be provided. When the rate of dissolution is 0.0005 Å/sec or more, resolution performance may also be enhanced. It is presumed that the reason is because the change in solubility before and after exposure of the above polymer or compound leads to an increase in contrast at the interface between an exposed region soluble in the developer and an unexposed region insoluble in the developer. The effects of reducing line edge roughness and of reducing defects are also exerted.

In a case where the material composition for lithography corresponds to a negative resist pattern, the rate of dissolution at 23° C. of an amorphous film formed by spin coating with the material composition for lithography, in a developer, is preferably 10 Å/sec or more. When the rate of dissolution is 10 Å/sec or more, the material composition is easily soluble in the developer and is much more suitable for a resist. When the rate of dissolution is 10 Å/sec or more, resolution performance may also be enhanced. It is presumed that the reason is because a micro surface portion of the polymer or the compound is dissolved to result in a reduction in line edge roughness. The effect of reducing defects is also exerted. The rate of dissolution can be determined by dipping the amorphous film in the developer at 23° C. for a predetermined time, and measuring the film thickness before and after the dipping, visually or by a known method such as an ellipsometer or a QCM method.

In a case where the material composition for lithography corresponds to a positive resist pattern, the rate of dissolution at 23° C. of a region of an amorphous film formed by spin coating with the material composition for lithography, in a developer, the region being exposed by radiation from, for example, a KrF excimer laser, extreme ultraviolet light, electron beam or X-ray, is preferably 10 Å/sec or more. When the rate of dissolution is 10 Å/sec or more, the material composition is easily soluble in the developer and is much more suitable for a resist. When the rate of dissolution is 10 Å/sec or more, resolution performance may also be enhanced. It is presumed that the reason is because a micro surface portion of the polymer or the compound is dissolved to result in a reduction in line edge roughness. The effect of reducing defects is also exerted.

In a case where the material composition for lithography corresponds to a negative resist pattern, the rate of dissolution at 23° C. of a region of an amorphous film formed by spin coating with the material composition for lithography, in a developer, the region being exposed by radiation from, for example, a KrF excimer laser, extreme ultraviolet light, electron beam or X-ray, is preferably 5 Å/sec or less, more preferably 0.0005 to 5 Å/sec, further preferably 0.05 to 5 Å/sec. When the rate of dissolution is 5 Å/sec or less, a resist insoluble in the developer can be provided. When the rate of dissolution is 0.0005 Å/sec or more, resolution performance may also be enhanced. It is presumed that the reason is because the change in solubility before and after exposure of the polymer or the compound leads to an increase in contrast at the interface between an unexposed region soluble in the developer and an exposed region insoluble in the developer. The effects of reducing line edge roughness and of reducing defects are also exerted.

### (Other Component in Material Composition for Lithography)

The material composition for lithography includes the above polymer or compound as a solid component, and also a solvent. The solvent for use in the material composition for lithography is not particularly limited, and those disclosed in Patent Document 1 may be used. The solvent is preferably a safe solvent, more preferably at least one selected from PGMEA, PGME, CHN, CPN, 2-heptanone, anisole, butyl acetate, ethyl propionate and ethyl lactate, further preferably at least one selected from PGMEA, PGME and CHN.

The relationship between the amount of the solid component and the amount of the solvent in the material composition for lithography is not particularly limited, and is preferably as follows relative to 100 mass % of the total mass of the solid component and the solvent.
1 to 80 mass % of the solid component: 20 to 99 mass % of the solvent
1 to 50 mass % of the solid component: 50 to 99 mass % of the solvent
2 to 40 mass % of the solid component: 60 to 98 mass % of the solvent
2 to 10 mass % of the solid component: 90 to 98 mass % of the solvent

The material composition for lithography may include at least one selected from the group consisting of an acid generating agent (C), an acid crosslinking agent (G), an acid diffusion controlling agent (E) and other component (F), as other solid component.

The content of the polymer or compound (the sum, if both are used) in the material composition for lithography is not particularly limited, and is preferably 50 to 99.4 mass %, more preferably 55 to 90 mass %, further preferably 60 to 80 mass %, and particularly preferably 60 to 70 mass % based on the total mass of the solid component (the sum of the polymer, the compound, and the solid component(s) optionally used, for example, the acid generating agent (C), the acid crosslinking agent (G), the acid diffusion controlling agent (E) and other component (F), the same applies to the following). The above content results in a further enhancement in resolution and a further reduction in line edge roughness (LER).

### (Acid Generating Agent (C))

The material composition for lithography preferably includes at least one acid generating agent (C) which directly or indirectly generates an acid by irradiation with any radiation selected from visible light, ultraviolet light, excimer laser, electron beam, extreme ultraviolet light (EUV), X-ray and ion beam.

In this case, the content of the acid generating agent (C) in the material composition for lithography is preferably 0.001 to 49 mass %, more preferably 1 to 40 mass %, further preferably 3 to 30 mass %, particularly preferably 10 to 25 mass %, based on the mass of the total solid component. The acid generating agent (C) is used in the range of the content, and thus higher sensitivity is achieved and a pattern profile lower in edge roughness is obtained.

The acid generation method is not limited as long as an acid is generated in the system in the material composition for lithography. If excimer laser is used instead of ultraviolet light such as g-ray or i-ray, finer processing can be made, and if electron beam, extreme ultraviolet light, X-ray or ion beam is used as a high energy line, further finer processing can be made.

The acid generating agent (C) is not particularly limited, and examples thereof include any compound disclosed in International Publication No. WO2017/033943. The acid generating agent (C) is preferably an acid generating agent having an aromatic ring, more preferably an acid generating agent having an sulfonic acid ion having an aryl group, particularly preferably diphenyltrimethylphenylsulfonium p-toluenesulfonate, triphenylsulfonium p-toluenesulfonate, triphenylsulfonium trifluoromethanesulfonate, or triphenylsulfonium nonafluoromethanesulfonate. Use of the acid generating agent can result in a reduction in line edge roughness.

It is preferable that the material composition for lithography further includes the diazonaphthoquinone optically active compound disclosed in Patent Literature 1 as the acid generating agent. In particular, an optically active non-polymeric diazonaphthoquinone compound is preferable, a low molecular compound having a molecular weight of 1500 or less is more preferable, a low molecular compound having a molecular weight of 1200 or less is further preferable, and a low molecular compound having a molecular weight of 1000 or less is particularly preferable, from the viewpoints of low roughness and the solubility. Preferable specific examples of the optically active non-polymeric diazonaphthoquinone compound include any optically active non-polymeric diazonaphthoquinone compound disclosed in International Publication No. WO2016/158881. The acid generating agent (C) can be used singly or in combinations of two or more kinds thereof.

### (Acid Crosslinking Agent (G))

In a case where the material composition for lithography is used as a negative resist material or is used as an additive for an increase in strength of a pattern even in the case of a positive resist material, it preferably includes at least one acid crosslinking agent (G). The acid crosslinking agent (G) is a compound which can intramolecularly or intermolecularly crosslink the polymer or compound in the presence of an acid generated from the acid generating agent (C). The acid crosslinking agent (G) is not particularly limited, and examples thereof can include a compound having at least one group which can crosslink the polymer or compound (hereinafter referred to as a “crosslinkable group”).

Specific examples of the crosslinkable group can include, but are not particularly limited to, those disclosed in Patent Document 1. The crosslinkable group in the acid crosslinking agent (G) is preferably a hydroxyalkyl group or an alkoxyalkyl group, particularly preferably an alkoxymethyl group.

Examples of the crosslinking agent (G) having a crosslinkable group can include, but are not particularly limited to, those disclosed in Patent Document 1.

A compound having a phenolic hydroxy group, and a compound and a resin each obtained by introducing the crosslinkable group into an acidic functional group in an alkali-soluble resin to thereby impart crosslinkability disclosed in Patent Document 1 can be further used as the acid crosslinking agent (G).

The acid crosslinking agent (G) in the material composition for lithography is preferably an alkoxyalkylated urea compound or a resin thereof, or an alkoxyalkylated glycoluryl compound or a resin thereof (acid crosslinking agent (G1)), a phenol derivative having 1 to 6 benzene rings in its molecule and having two or more hydroxyalkyl groups or alkoxyalkyl groups in its molecule, in which the hydroxyalkyl groups or alkoxyalkyl groups are bound to any benzene ring described above (acid crosslinking agent (G2)), or a compound having at least one α-hydroxyisopropyl group (acid crosslinking agent (G3)). Examples include any compound disclosed in International Publication No. WO2017/033943.

The content of the acid crosslinking agent (G) in the material composition for lithography is preferably 0.5 to 49 mass %, more preferably 0.5 to 40 mass %, further preferably 1 to 30 mass %, particularly preferably 2 to 20 mass %, based on the mass of the total solid component. The content ratio of the acid crosslinking agent (G) is preferably 0.5 mass % or more because of enabling the effect of suppressing solubility of a resist film in an alkaline developing solution to be enhanced, and enabling a reduction in rate of the residual film, and the occurrence of swelling and meandering of a pattern to be suppressed, and on the other hand, the content ratio is preferably 49 mass % or less because of enabling deterioration in heat resistance of a resist to be suppressed.

The content of at least one selected from the acid crosslinking agent (G1), the acid crosslinking agent (G2) and the acid crosslinking agent (G3) in the acid crosslinking agent (G) is also not particularly limited, and can fall within various ranges depending on the type and the like of the substrate for use in resist pattern formation.

### (Acid Diffusion Controlling Agent (E))

The material composition for lithography may include an acid diffusion controlling agent (E) which has the effect of controlling diffusion of an acid generated from the acid generating agent by irradiation with radiation, in a resist film, to thereby inhibit an undesirable chemical reaction in an unexposed region. The acid diffusion controlling agent (E) is used to result in an enhancement in preservation stability of the material composition for lithography. Additionally, not only the resolution is further enhanced, but also a resist pattern can be inhibited from being changed in line width due to the variations in post exposure delay before irradiation with radiation and post exposure delay after irradiation with radiation, and is extremely excellent in process stability.

The acid diffusion controlling agent (E) is not particularly limited, and examples thereof include radiation-degradable basic compounds such as a nitrogen atom-containing basic compound, a basic sulfonium compound and a basic iodonium compound. Examples of the acid diffusion controlling agent (E) include any compound disclosed in International Publication No. WO2017/033943. The acid diffusion controlling agent (E) can be used singly or in combinations of two or more kinds thereof.

The content of the acid diffusion controlling agent (E) is preferably 0.001 to 49 mass %, more preferably 0.01 to 10 mass %, further preferably 0.01 to 5 mass %, particularly preferably 0.01 to 3 mass %, based on the mass of the total solid component. When the content of the acid diffusion controlling agent (E) is in the above range, a reduction in resolution, and degradation of a pattern shape, dimensional faithfulness and the like can be further suppressed. Furthermore, even if the post exposure delay after irradiation with radiation from irradiation with electron beam is increased, the shape of the upper layer portion of a pattern is not degraded. When the content of the acid diffusion controlling agent (E) is 10 mass % or less, sensitivity, developability in an unexposed region, and the like can be prevented from being deteriorated. The acid diffusion controlling agent is used to not only result in an enhancement in preservation stability of the material composition for lithography and an enhancement in resolution, but also enable a resist pattern to be inhibited from being changed in line width due to the variations in post exposure delay before irradiation with radiation and post exposure delay after irradiation with radiation, and to be extremely excellent in process stability.

### (Other Component (F))

One or more of various additives such as a dissolution promoting agent, a dissolution controlling agent, a sensitizing agent, a surfactant, and an organic carboxylic acid or an oxo acid of phosphorus, or any derivative thereof can be, if necessary, added as other component (F) to the material composition for lithography as long as the objects of the present embodiment are not impaired. Examples of such other component (F) include any compound disclosed in International Publication No. WO2017/033943.

The total content of such other component (F) is preferably 0 to 49 mass %, more preferably 0 to 5 mass %, further preferably 0 to 1 mass %, particularly preferably 0 mass %, based on the mass of the total solid component.

In the material composition for lithography, the content of the polymer or compound, the acid generating agent (C), the acid diffusion controlling agent (E) and other component (F): (the polymer or compound (the sum, if both are used)/the acid generating agent (C)/the acid diffusion controlling agent (E)/other component (F)), is as follows, as expressed by mass % on the solid content basis.
preferably 50 to 99.4/0.001 to 49/0.001 to 49/0 to 49
more preferably 55 to 90/1 to 40/0.01 to 10/0 to 5
further preferably 60 to 80/3 to 30/0.01 to 5/0 to 1
particularly preferably 60 to 70/10 to 25/0.01 to 3/0

The content ratio among the respective components is selected from such various ranges so that the sum of the components is 100 mass %. The above content ratio allows performances such as sensitivity, resolution, and developability to be further excellent.

In an embodiment, the material composition for lithography includes the polymer or compound, the acid generating agent (C) and the acid crosslinking agent (G). The preferred amount of the respective components in this case are as follows as expressed by mass % on the solid content basis.
preferably 30 to 99.4/0.001 to 49/0.001 to 49
more preferably 35 to 90/1 to 40/1 to 40
further preferably 40 to 80/1.5 to 30/2 to 30
particularly preferably 45 to 70/2 to 20/2 to 20

In this case the material composition for lithography may include 0.001 to 2 mass % of the acid diffusion controlling agent (E) based on the solid content.

The method for preparing the material composition for lithography is not particularly limited, and examples thereof include a method including dissolving the respective components in the solvent in use to thereby provide a homogeneous solution, and thereafter, if necessary, filtering the solution by, for example, a filter having a pore size of about 0.2 μm.

The material composition for lithography can include a resin as long as the objects of the present invention are not impaired. The resin is not particularly limited, and examples thereof include a novolac resin, polyvinylphenols, polyacrylic acid, polyvinylalcohol, a styrene-maleic anhydride resin, and a polymer containing acrylic acid, vinylalcohol or vinylphenol as a monomer unit, or any derivative thereof. The content of the resin is not particularly limited, and is adjusted depending on the type of the polymer or compound. The content is preferably 30 parts by mass or less, more preferably 10 parts by mass or less, further preferably 5 parts by mass or less, and particularly preferably 0 parts by mass based on 100 parts by mass of the compound.

### [Method for Forming Pattern]

In a case where a pattern is formed on a substrate by use of a material for lithography, for example, a method for forming a pattern can be used which includes a film formation step of forming a film on a substrate by use of the material for lithography according to the present embodiment or a composition including the material (hereinafter, these may be sometimes collectively referred to as “material or the like for lithography”), an exposure step of exposing the film, and a development step of developing the film exposed in the exposure step, to thereby form a pattern.

For example, in a case where the material or the like for lithography is used to form a resist pattern, a method for forming a pattern (resist pattern) is not particularly limited, and examples of a suitable method include a method including a film formation step of coating a substrate with a resist composition including the above material or the like for lithography to thereby form a film (resist film), an exposure step of exposing the film (resist film) formed, and a development step of developing the film (resist film) exposed in the exposure step to thereby form a pattern (resist pattern). The resist pattern in the present embodiment can also be formed as an upper layer resist in a multi-layer process.

Examples of a specific method for forming the resist pattern include, but not particularly limited, the following method. First, a conventionally known substrate is coated with the resist composition by a coating procedure such as rotation coating, cast coating, or roll coating to thereby form a resist film. The conventionally known substrate is not particularly limited, and examples thereof include a substrate for electronic components, and such a substrate on which a predetermined wiring pattern is formed. More specific examples include, but not particularly limited, a silicon wafer, substrates made of metals such as copper, chromium, iron, and aluminum, and a glass substrate. Examples of the material for the wiring pattern include, but not particularly limited, copper, aluminum, nickel, and gold. An inorganic film or an organic film may be, if necessary, provided on the above substrate. Examples of the inorganic film include, but not particularly limited, an inorganic antireflective film (inorganic BARC). Examples of the organic film include, but not particularly limited, an organic antireflective film (organic BARC). A surface treatment with hexamethylene disilazane or the like may also be performed.

Next, the substrate coated is, if necessary, heated. The heating condition, while varied depending on the compositional profile of the resist composition, and the like, is preferably 20 to 250° C., more preferably 20 to 150° C. Such heating is preferable because of sometimes resulting in an enhancement in close contact of the resist with the substrate. Next, the resist film is exposed to any radiation selected from the group consisting of visible light, ultraviolet light, excimer laser, electron beam, extreme ultraviolet light (EUV), X-ray and ion beam, to thereby provide a desired pattern. The exposure conditions and the like are appropriately selected depending on the compositional profile of compounding in the resist composition, and the like. In the method for forming a resist pattern, heating is preferably performed after irradiation with radiation in order to stably form a high-accuracy and fine pattern in exposure. The heating condition, while varied depending on the compositional profile of compounding in the resist composition, and the like, is preferably 20 to 250° C., more preferably 20 to 150° C.

Next, the resist film exposed is developed by a developer to thereby form a predetermined resist pattern. It is preferable to select a solvent having a solubility parameter (SP value) closer to that of the polymer or compound as the developer. Preferred examples of solvents are those disclosed in Patent Document 1.

A proper amount of a surfactant can be, if necessary, added to the developer. The surfactant is not particularly limited, and, for example, an ionic or non-ionic fluorine-based or silicon-based surfactant can be used. Examples of these surfactants are those disclosed in Patent Document 1. It is more preferable to use a fluorine-based surfactant or a silicon-based surfactant as the non-ionic surfactant.

The amount of the surfactant used is usually 0.001 to 5 mass %, preferably 0.005 to 2 mass %, further preferably 0.01 to 0.5 mass %, based on the amount of the entire developer.

The development method here applied can be, for example, a method (dipping method) including dipping the substrate in a tank filled with the developer, for a certain time, a method (paddle method) including raising the developer on the substrate surface by surface tension and leaving it to still stand for a certain time for development, a method (spraying method) including spraying the developer on the substrate surface, or a method (dynamic dispense method) including continuously discharging the developer onto the substrate rotating at a certain speed, with scanning of a developer discharge nozzle at a certain speed. The time for pattern development is not particularly limited, and is preferably 10 seconds to 90 seconds. After the development step, a step of stopping development under replacement with other solvent may be performed.

A step of washing with a rinsing liquid including an organic solvent, after development, is preferably included. The rinsing liquid for use in a rinsing step after development and the method of rinsing may be those disclosed in Patent Document 1.

After a resist pattern is formed, a pattern wiring substrate is obtained by etching. The etching method can be performed by a known method such as dry etching using plasma gas or wet etching with, for example, an alkali solution, a cupric chloride solution or a ferric chloride solution.

After a resist pattern is formed, plating can also be performed. Examples of the plating method include, but not particularly limited, copper plating, solder plating, nickel plating, and gold plating.

The remaining resist pattern after etching can be peeled by an organic solvent. The organic solvent and the peeling method may be those disclosed in Patent Document 1.

The wiring substrate can also be formed by a method including forming the resist pattern, then depositing a metal in vacuum and thereafter dissolving the resist pattern by a solution, namely, a liftoff method.

### [Composition for Resist Underlayer Film Formation, Underlayer Film for Lithography, and Method for Forming Pattern]

### First Embodiment

### <Composition for Resist Underlayer Film Formation>

The composition for resist underlayer film formation according to the present embodiment includes the polymer or compound and a silicon-containing compound (for example, a hydrolyzable organosilane, a hydrolysate thereof or a hydrolysis condensate thereof). The composition for resist underlayer film formation has relatively high carbon concentration, relatively low oxygen concentration, high heat resistance and also high solvent solubility. Thus, rectangularity of a pattern is excellent. The composition for resist underlayer film formation can reduce film defects (achieve thin film formation), has good storage stability, is highly sensitive, has high refractive index and is transparent in specific wavelength ranges and can provide a favorable resist pattern shape.

The composition for resist underlayer film formation can be suitably used in, for example, a method with a multi-layer resist where a resist underlayer film is further provided between an upper layer resist (a photoresist or the like) and a hard mask or an organic underlayer film. In such a multi-layer resist method, for example, a resist underlayer film is formed on an organic underlayer film or a hard mask interposed on the substrate, by a coating method or the like, and an upper layer resist (for example, a photoresist, an electron beam resist, or an EUV resist) is formed on the resist underlayer film. A resist pattern is formed by exposure and development, the resist underlayer film is dry etched by use of the resist pattern, to thereby transfer the pattern, and the organic underlayer film is etched to thereby transfer the pattern and process the substrate by the organic underlayer film.

In other words, the resist underlayer film (Underlayer film for lithography) formed by use of the composition for resist underlayer film formation not only hardly causes intermixing with the upper layer resist, but also has heat resistance and is higher in etching rate against, for example, a halogen-based (fluorine-based) etching gas, than the upper layer resist patterned, for use as a mask, and thus can provide a rectangular and favorable pattern. Furthermore, the resist underlayer film formed by use of the composition for resist underlayer film formation is high in resistance against an oxygen-based etching gas, and thus can serve as a favorable mask in patterning of a layer provided on a base material, such as a hard mask. The composition for resist underlayer film formation can also be used in a mode where a plurality of such resist underlayer films are laminated. In this case, such resist underlayer films formed by use of the composition for resist underlayer film formation are not particularly limited in terms of the location thereof (the orders of such films laminated), and may be located immediately below the upper layer resist, may be layers located closest to the substrate, or may be in a mode where sandwiching between such resist underlayer films is made.

When a fine pattern is formed, a resist film thickness tends to be thinner in order to prevent pattern collapse. A resist film is thinner, and thus pattern transfer can be made only when dry etching for transferring a pattern to a film present as an underlayer exhibits a higher etching rate than the etching rate of a film as an upper layer. In the present embodiment, the organic underlayer film can be interposed on the substrate and can be covered with the resist underlayer film (containing a silicon-based compound) of the present embodiment, and the resultant can be further covered with a resist film (organic resist film). An organic component film and an inorganic component film are considerably different in dry etching rate due to selection of an etching gas, and the organic component film is increased in dry etching rate by an oxygen-based gas and the inorganic component film is increased in dry etching rate by a halogen-containing gas.

For example, a resist underlayer film where pattern transfer is made can be used for dry etching of an organic underlayer film as an underlayer by an oxygen-based gas to thereby perform pattern transfer to the organic underlayer film, and the organic underlayer film where pattern transfer is made can be used for processing of the substrate by use of a halogen-containing gas.

The resist underlayer film with the composition for resist underlayer film formation includes the above Te-containing polymer or compound having excellent absorption ability of active light, and a silicon-containing compound (for example, a hydrolyzable organosilane, a hydrolysate thereof or a hydrolysis condensate thereof), thereby resulting in an enhancement in sensitivity of the upper layer resist, causing no intermixing with the upper layer resist, and allowing the shape of a pattern on the film forming the resist underlayer film after exposure and development to be rectangular. Thus, substrate processing by a fine pattern is made possible.

The resist underlayer film with the composition for resist underlayer film formation of the present embodiment has high heat resistance and thus can be used even in high-temperature baking conditions. Furthermore, the molecular weight is relatively low and the viscosity is low, thus even a substrate having difference in level (in particular, a fine space and/or a hole pattern) can be filled uniformly in its every corner, resulting in a tendency to relatively advantageously increase flattening properties and embedding properties.

The composition for resist underlayer film formation can further include a solvent, an acid, an acid crosslinking agent, and the like, in addition to the above polymer or compound and the silicon-containing compound. Furthermore, optional components such as an organic polymer compound, an acid generating agent and a surfactant, and other components such as water, an alcohol, and a curing catalyst can be included. The content of the compound and the resin according to the present embodiment in the composition for resist underlayer film formation is preferably 0.1 to 70 mass %, more preferably 0.5 to 50 mass % and particularly preferably 3.0 to 40 mass % from the viewpoints of coatability and quality stability.

A known solvent can be appropriately used as the solvent as long as it dissolves at least the polymer or compound. The type and the amount of the solvent may be as disclosed in Patent Document 1.

The composition for resist underlayer film formation can include an acid from the viewpoint of promotion of curability. The type and the amount of the acid may be as disclosed in Patent Document 1.

In a case where the composition for resist underlayer film formation is used as a negative resist material or is used as an additive for an increase in strength of a pattern even in the case of a positive resist material, the resist underlayer film forming composition can include at least one acid crosslinking agent. The acid crosslinking agent is a compound which can intramolecularly or intermolecularly crosslink the above polymer or compound in the presence of the acid described above. The type and the amount of the acid crosslinking agent may be as disclosed in Patent Document 1.

The composition for resist underlayer film formation includes a silicon-containing compound. The silicon-containing compound may be an organic silicon-containing compound or an inorganic silicon-containing compound, and is preferably an organic silicon-containing compound. The type and the amount of the silicon-containing compound may be as disclosed in Patent Document 1.

The composition for resist underlayer film formation can further include an organic polymer compound, a cross-linking agent, a photoacid generator, a surfactant and the like, if necessary, in addition to the above components. The type and the amount of these materials may be as disclosed in Patent Document 1.

### <Underlayer Film for Lithography and Method for Forming Pattern>

An underlayer film for lithography according to the present embodiment can be formed by use of the composition for resist underlayer film formation. The underlayer film for lithography of the present embodiment can be suitably used as an underlayer (resist underlayer film) of a photoresist (upper layer) for use in a multi-layer resist method. In the present embodiment, a pattern can be formed by, for example, forming a resist underlayer film by use of the composition for resist underlayer film formation, forming at least one photoresist layer on the resist underlayer film, and thereafter irradiating a predetermined region of the photoresist layer with radiation to thereby perform development.

One aspect of the method for forming a pattern according to the first embodiment of the present invention by use of the composition for resist underlayer film formation according to the first embodiment, produced as described above, can provide, for example, a method for forming a pattern, including forming an organic underlayer film on a substrate, with a coating type organic underlayer film material, forming a resist underlayer film on the organic underlayer film, by use of the composition for resist underlayer film formation of the first embodiment of the present invention, forming an upper layer resist film on the resist underlayer film, by use of an upper layer resist film composition, forming an upper layer resist pattern on the upper layer resist film, transferring a pattern to the resist underlayer film by etching with the upper layer resist pattern as a mask, transferring a pattern to the organic underlayer film by etching with the resist underlayer film to which a pattern is transferred, as a mask, and further transferring a pattern to the substrate (object to be processed) by etching with the organic underlayer film to which a pattern is transferred, as a mask.

Another aspect of the method for forming a pattern according to the first embodiment can provide, for example, a method for forming a pattern, including forming an organic hard mask containing carbon as a main component, on a substrate, by a CVD method, forming a resist underlayer film on the organic hard mask, by use of the composition for resist underlayer film formation of the first embodiment, forming an upper layer resist film on the resist underlayer film, by use of an upper layer resist film composition, forming an upper layer resist pattern on the upper layer resist film, transferring a pattern to the resist underlayer film by etching with the upper layer resist pattern as a mask, transferring a pattern to the organic hard mask by etching with the resist underlayer film to which a pattern is transferred, as a mask, and further transferring a pattern to the base material (object to be processed) by etching with the organic hard mask to which a pattern is transferred, as a mask. The base material here used can be, for example, a semiconductor substrate. A silicon substrate can be commonly used as the semiconductor substrate, and any substrate of Si, amorphous silicon (α-Si), p-Si, SiO₂, SiN, SiON, W, TiN, Al, or the like, different in material from a layer to be processed, can be used without any particular limitation. Those disclosed in Patent Document 1 may be used as the metal constituting the substrate (object to be processed; including the semiconductor substrate).

An organic underlayer film or an organic hard mask can be formed on the substrate in the method for forming a pattern of the present embodiment. In particular, the organic underlayer film can be formed from a coating type organic underlayer film material, by a rotation coating method or the like, and the organic hard mask can be formed from a material of an organic hard mask containing carbon as a main component, by a CVD method. The organic underlayer film and the organic hard mask are not particularly limited in terms of the types thereof, and are each preferably one which exhibits a sufficient antireflective film function in a case where the upper layer resist film is exposed to thereby form a pattern. The organic underlayer film or the organic hard mask can be formed to thereby transfer the pattern formed based on the upper layer resist film, onto the base material (object to be processed), without the occurrence of any difference in size conversion. Herein, the hard mask “containing carbon as a main component” means a hard mask in which 50 mass % or more of the solid content is constituted from a carbon-based material of amorphous hydrogenated carbon or the like called also “amorphous carbon” and represented by a —C:H. While an a —C:H film can be deposited by various techniques, plasma-enhanced chemical vapor deposition (PECVD) is widely used to achieve cost efficiency and to enable film quality to be adjusted. Examples of the hard mask can be seen in those described in, for example, Japanese Translation of PCT International Application Publication No. 2013-526783.

The resist underlayer film with the composition for resist underlayer film formation, for use in the method for forming a pattern of the present embodiment, can be produced on an object to be processed, on which the organic underlayer film or the like is provided from the composition for resist underlayer film formation, by a spin coating method or the like. In a case where the resist underlayer film is formed by a spin coating method, baking is desirably made after spin coating in order to evaporate a solvent and promote a crosslinking reaction for the purpose of prevention of mixing with the upper layer resist film. The baking temperature is preferably in the range from 50 to 500° C. The baking temperature is here particularly preferably 400° C. or less in order to decrease thermal damage to the device, while depends on the structure of a device to be produced. The baking time is preferably in the range from 10 seconds to 300 seconds.

The method for forming a pattern on the upper layer resist film, adopted in the method for forming a pattern of the present embodiment, can be suitably any method of a lithography method using light at a wavelength of 300 nm or less or EUV light; an electron beam direct drawing method, and an inductive self-organization method. Such a method can be used to thereby form a fine pattern on the upper layer resist film.

The upper layer resist film composition can be appropriately selected depending on the method for forming a pattern on the upper layer resist film. For example, in the case of lithography with light of 300 nm or less, or EUV light, a chemical amplification type photoresist film material can be used as the upper layer resist film composition. Examples of such a photoresist film material can include a material for forming a positive pattern by formation of a photoresist film and exposure thereof, and then dissolution of an exposed region with an alkaline developing solution, and a material for forming a negative pattern by such formation and exposure and then dissolution of an unexposed region with a developer including an organic solvent.

A resist underlayer film formed from the composition for resist underlayer film formation of the present embodiment may absorb the light, while depends on the wavelength of light for use in a lithography process. In this case, the film can function as an antireflective film having the effect of preventing light reflected from the substrate.

An EUV resist underlayer film not only functions as a hard mask, but also can be used for the following purpose. The above Te-containing composition for resist underlayer film formation can be used as an underlayer antireflective film of EUV resist, which does not intermix with the EUV resist and can prevent reflection of undesirable exposure light in EUV exposure (wavelength 13.5 nm), for example, the above UV and DUV (ArF light, KrF light), from the substrate or interface. Such an underlayer of EUV resist can efficiently prevent reflection. The composition for underlayer film formation is excellent in absorption ability of EUV, and thus can exhibit sensitization of an upper layer resist composition, and contributes to an enhancement in sensitivity. In the case of use in such an EUV resist underlayer film, a process as in that of an underlayer film for photoresists can be performed.

### Second Embodiment

### <Composition for Resist Underlayer Film Formation>

The composition for resist underlayer film formation according to the second embodiment can reduce film defects (achieve thin film formation), has good storage stability, is highly sensitive, has high refractive index and is transparent in specific wavelength ranges and can provide a favorable resist pattern shape. The composition for resist underlayer film formation may not include a silicon-containing compound.

The composition for resist underlayer film formation according to the present embodiment can be applied to a wet process, and can form a resist underlayer film which has excellent resolution and sensitivity and achieves excellent resist pattern after development. Furthermore, the composition for resist underlayer film formation is useful for forming a photoresist underlayer film having excellent heat resistance, level embedding properties and flatness. The composition for resist underlayer film formation uses a compound that has a specified structure and relatively high carbon concentration, relatively low oxygen concentration and high solvent solubility, and thus an underlayer film not only suppressed in degradation during baking, but also excellent in etching resistance to fluorine gas-based plasma etching or the like can be formed. Furthermore, close contact with a resist layer is also excellent, and thus an excellent resist pattern can be formed. The composition for resist underlayer film formation of the present embodiment particularly has excellent heat resistance and level embedding properties, and provides excellent flatness, and thus, for example, can be used as a composition for forming a resist underlayer film provided as the undermost layer among a plurality of resist layers. Herein, the resist underlayer film formed by use of the composition for resist underlayer film formation of the present embodiment may further include other resist underlayer between the film and the substrate.

The composition for resist underlayer film formation according to the present embodiment may further include a solvent, an acid generating agent, an acid crosslinking agent and the like. Furthermore, optional components such as a basic compound and other components such as water, an alcohol, and a curing catalyst can be included. The content of the polymer or compound (the sum, if both are used) in the composition for resist underlayer film formation is preferably 0.1 to 70 mass %, more preferably 0.5 to 50 mass % and particularly preferably 3.0 to 40 mass % from the viewpoints of coatability and quality stability.

A known solvent can be appropriately used as the solvent for use in the present embodiment as long as it dissolves at least the polymer or compound. The type and the amount of the solvent may be as disclosed in Patent Document 1.

The composition for resist underlayer film formation of the present embodiment may, if necessary, include an acid crosslinking agent from the viewpoint of, for example, suppression of intermixing. The type and the amount of the acid crosslinking agent may be as disclosed in Patent Document 1.

The composition for resist underlayer film formation according to the present embodiment may further include an acid generating agent as needed from the viewpoint of acceleration of cross-linking reaction by heat. The type and the amount of the acid generating agent may be as disclosed in Patent Document 1.

The composition for resist underlayer film formation according to the present embodiment may further include a basic compound from the viewpoint of improvement in storage stability. The type and the amount of the basic compound may be as disclosed in Patent Document 1.

The composition for resist underlayer film formation according to the present embodiment may further include another resin or compound for the purposes of imparting thermosetting properties and controlling absorbance. Those disclosed in Patent Document 1 may be used as another resin or compound.

### <Resist Underlayer Film for Lithography and Method for Forming Pattern>

A resist underlayer film for lithography according to the present second embodiment is formed by use of the composition for resist underlayer film formation according to the second embodiment. A pattern formed in the present embodiment can be used as, for example, a resist pattern or a circuit pattern.

A method for forming a pattern according to the second embodiment includes a step (step A-1) of forming a resist underlayer film on a substrate, by use of the composition for resist underlayer film formation of the second embodiment of the present invention, a step (step A-2) of forming at least one photoresist layer on the resist underlayer film, and a step (step A-3) of, after formation of the at least one photoresist layer in step A-2, irradiating a predetermined region of the photoresist layer with radiation to thereby perform development. The “photoresist layer” means a layer provided in the outermost layer of the resist layer, namely, the outermost surface of the resist layer (opposite to the substrate).

Other method for forming a pattern of the second embodiment of the present invention includes a step (step B-1) of forming a resist underlayer film on a substrate, by use of the composition for resist underlayer film formation of the second embodiment of the present invention, a step (step B-2) of forming a resist intermediate layer film on the underlayer film, by use of a resist intermediate layer film material (for example, silicon-containing resist layer), a step (step B-3) of forming at least one photoresist layer on the resist intermediate layer film, a step (step B-4) of, after formation of the at least one photoresist layer in step B-3, irradiating a predetermined region of the photoresist layer with radiation to thereby perform development and thus form a resist pattern, and a step (step B-5) of, after formation of the resist pattern in step B-4, etching the resist intermediate layer film with the resist pattern as a mask, etching the underlayer film with the resulting intermediate layer film pattern, as an etching mask, and etching the substrate with the resulting underlayer film pattern as an etching mask to thereby form a pattern on the substrate.

The method for forming the resist underlayer film for lithography of the present embodiment is not particularly limited as long as the resist underlayer film is formed from the composition for resist underlayer film formation of the present embodiment, and a known procedure can be applied. For example, the resist underlayer film can be formed by applying the composition for resist underlayer film formation of the present embodiment to a substrate by a known coating method or printing method such as spin coating or screen printing, and thereafter removing an organic solvent by volatilization or the like.

The resist underlayer film, when formed, is preferably subjected to baking treatment in order to not only suppress the occurrence of a mixing phenomenon with an upper layer resist (for example, a photoresist layer or a resist intermediate layer film), but also promote a crosslinking reaction. The condition may be as disclosed in Patent Document 1.

After the resist underlayer film is produced on the substrate, a resist intermediate layer film can be provided between the photoresist layer and the resist underlayer film. For example, in the case of a two-layer process, a silicon-containing resist layer, a single-layer resist including usual hydrocarbon, or the like can be provided as a resist intermediate layer film on the resist underlayer film. For example, in the case of a three-layer process, preferably, a silicon-containing intermediate layer is produced between the resist intermediate layer film and the photoresist layer, and furthermore a single-layer resist layer containing no silicon is produced thereon. The photoresist materials for use in formation of the photoresist layer, the resist intermediate layer film, and the resist layer provided therebetween can be known photoresist materials. Those disclosed in Patent Document 1 may be used as the silicon-containing resist material. A resist intermediate layer film formed by a Chemical Vapour Deposition (CVD) method can also be used.

A resist underlayer film of the present embodiment can also be used as an antireflective film for a usual single-layer resist or as an underlying material for suppression of pattern collapse. The resist underlayer film of the present embodiment is excellent in etching resistance for underlying processing, and thus can also be expected to function as a hard mask for underlying processing.

In a case where a resist layer is formed by the above known photoresist material, a wet process such as a spin coating method or screen printing is preferably used as in the case of formation of the resist underlayer film. After coating with the resist material according to a spin coating method or the like, pre-baking is usually performed, and the pre-baking is preferably performed at a baking temperature ranging from 80 to 180° C. for a baking time ranging from 10 seconds to 300 seconds. Thereafter, exposure can be performed and post-exposure baking (PEB) and development can be performed according to ordinary methods, to thereby provide a resist pattern. The thickness of each resist film is not particularly limited, and is generally preferably 30 nm to 500 nm, more preferably 50 nm to 400 nm.

The exposure light may be appropriately selected and used depending on the photoresist material used. Examples can commonly include high energy line having a wavelength of 300 nm or less, specifically, excimer laser at 248 nm, 193 nm and 157 nm, soft X-ray at 3 to 20 nm, electron beam, and X-ray.

A resist pattern formed by the above method is suppressed in pattern collapse by the resist underlayer film of the present embodiment. Therefore, the resist underlayer film of the present embodiment can be used to thereby obtain a finer pattern and also reduce the amount of exposure necessary for obtaining such a resist pattern.

Next, the resulting resist pattern is used as a mask to perform etching. Gas etching is preferably used as etching of the resist underlayer film in a two-layer process. Gas etching is suitably etching using an oxygen gas. Not only an oxygen gas, but also an inert gas such as He or Ar, and/or CO, CO₂, NH₃, SO₂, N₂, NO₂, and/or H₂ gas(es) can also be added. Gas etching can also be performed without use of any oxygen gas by using only CO, CO₂, NH₃, SO₂, N₂, NO₂, and/or H₂ gas(es). In particular, the latter gas(es) is/are preferably used for side wall protection for prevention of undercutting of a pattern side wall.

Gas etching is preferably used also as etching of the intermediate layer (the layer located between the photoresist layer and the resist underlayer film) in a three-layer process. Gas etching here applied can be the same as that described above with respect to a two-layer process. In particular, processing of the intermediate layer in a three-layer process is preferably performed using a fluorocarbon gas with a resist pattern as a mask. Thereafter, for example, oxygen gas etching can be performed with an intermediate layer pattern as a mask, as described above, thereby performing processing of the resist underlayer film.

In a case where an inorganic hard mask intermediate layer film is formed as the intermediate layer, a silicon oxide film, a silicon nitride film, or a silicon oxide nitride film (SiON film) is formed by a CVD method, an ALD method, or the like. The method for forming a nitride film can be, but are not limited to, for example, any method described in Japanese Patent Laid-Open No. 2002-334869 or International Publication No. WO2004/066377. While a photoresist film can be formed directly on such an intermediate layer film, a photoresist film may be formed on an organic antireflective film (BARC) formed on such an intermediate layer film by spin coating.

A polysilsesquioxane-based intermediate layer is also preferably used as the intermediate layer. A resist intermediate layer film can be allowed to have the effect of an antireflective film, to result in a tendency to effectively suppress reflection. A specific material of the polysilsesquioxane-based intermediate layer, here used, can be, but are not limited to, any material described in, for example, Japanese Patent Laid-Open No. 2007-226170 or Japanese Patent Laid-Open No. 2007-226204.

Etching of the substrate can also be performed by an ordinary method, and, for example, etching mainly with a fluorocarbon gas can be performed in a case where the substrate is made of SiO₂ or SiN, and etching mainly with a chlorine-based or bromine-based gas can be performed in a case where the substrate is made of p-Si, Al, or W. In a case where the substrate is etched with a fluorocarbon gas, the silicon-containing resist in a two-layer resist process and the silicon-containing intermediate layer in a three-layer process are peeled at the same time as processing of the substrate. In a case where the substrate is etched with a chlorine-based or bromine-based gas, the silicon-containing resist layer or the silicon-containing intermediate layer is separately peeled, generally, peeled by dry etching with a fluorocarbon gas after processing of the substrate.

The resist underlayer film of the present embodiment is excellent in etching resistance of the substrate. The substrate can be appropriately selected from known substrates and used, and is not particularly limited, and examples thereof include Si, α-Si, p-Si, SiO₂, SiN, SiON, W, TiN, and Al. The substrate may be a laminate having on a base material (support), a film to be processed (substrate to be processed). Examples of the film to be processed include various Low-k films of Si, SiO₂, SiON, SiN, p-Si, α-Si, W, W—Si, Al, Cu, Al—Si, and the like, and stopper films thereof, and any substrate whose material is different from the base material (support) is usually used. The thickness of the substrate of interest to be processed or the film to be processed is not particularly limited, and is usually preferably about 50 nm to 10,000 nm, more preferably 75 nm to 5,000 nm.

The resist underlayer film of the present embodiment is excellent in embedding flatness to a substrate having difference in level. A known evaluation method can be appropriately selected and used as the method for evaluating the embedding flatness, and the embedding flatness to a substrate having difference in level can be evaluated, without any particular limitation, by, for example, coating a silicon substrate having difference in level, with a solution of each compound having a predetermined concentration adjusted, by spin coating, performing drying and removal of a solvent at 110° C. for 90 seconds to thereby form a Te-containing underlayer film so that a predetermined thickness is achieved, and thereafter measuring the difference (ΔT) in underlayer film thickness between a line and space region and an opening region having no pattern after baking at a temperature of about 240 to 300° C. for a predetermined time, with an ellipsometer.

### (Composition for Optical Article Formation and Cured Product Thereof)

The composition for optical article formation according to the present embodiment is useful for optical materials. The composition for optical article formation according to the present embodiment includes the Te-containing polymer or compound described above, and thus is expected to have high refractive index and be highly transparent, and is also expected to have storage stability, structure-forming ability (film-forming ability) and heat resistance. The refractive index of the optical article is preferably 1.60 or more, more preferably 1.65 or more, even more preferably 1.70 or more, and further preferably 1.75 or more from the viewpoints of a reduction in size of an optical component and an enhancement in light collection rate. The transparency of the optical article is preferably 70% or more, more preferably 80% or more, further preferably 90% or more from the viewpoint of an enhancement in light collection rate. The method for measuring the refractive index is not particularly limited and a known method is used. Examples include a spectroscopic ellipsometry method, a minimum deviation method, a critical angle method (Abbe's system or Pulfrich's system), a V-block method, a prism coupler method, and an immersion method (Becke's line method). The method for measuring the transparency is not particularly limited and a known method is used. Examples include a method with a spectrophotometer and a spectroscopic ellipsometry method.

A cured product according to the present embodiment obtained by curing of the composition for optical component formation, can be a three-dimensionally crosslinked product, is suppressed in coloration by a heat treatment in a broad range from a low temperature to a high temperature, and can be expected to have a high refractive index and high transparency.

The composition for optical article formation according to the present embodiment may further include a solvent in addition to the above polymer or compound. The solvent may be the same as the solvent used for the material composition for lithography according to the present embodiment described above.

The relationship between the amount of the solid component and the amount of the solvent in the composition for optical component formation of the present embodiment is not particularly limited, and is preferably a relationship between 1 to 80 mass % of the solid component and 20 to 99 mass % of the solvent, more preferably 1 to 50 mass % of the solid component and 50 to 99 mass % of the solvent, further preferably 2 to 40 mass % of the solid component and 60 to 98 mass % of the solvent, particularly preferably 2 to 10 mass % of the solid component and 90 to 98 mass % of the solvent, based on 100 mass % of the total of the solid component and the solvent. The composition for optical component formation of the present embodiment can also include no solvent.

The composition for optical component formation of the present embodiment may include at least one selected from the group consisting of an acid generating agent (C), an acid crosslinking agent (G), an acid diffusion controlling agent (E) and other component (F), as other solid component.

The content of the polymer or compound (the sum, if both are used) in the composition for optical article formation according to the present embodiment is not particularly limited and is preferably 50 to 99.4% by mass, more preferably 55 to 90% by mass, further preferably 60 to 80% by mass, and particularly preferably 60 to 70% by mass based on the total mass of the solid component.

The acid generating agent (C), the acid crosslinking agent (G), the acid diffusion controlling agent (E) and other component (F) included in the composition for optical article formation according to the present embodiment may be the same as those optionally included in the material composition for lithography according to the present embodiment described above.

In the composition for optical article formation according to the present embodiment, the content of the polymer or compound, the acid generating agent (C), the acid diffusion controlling agent (E) and other component (F): (the polymer or compound (the sum, if both are used)/the acid generating agent (C)/the acid diffusion controlling agent (E)/other component (F)), is preferably 50 to 99.4/0.001 to 49/0.001 to 49/0 to 49, more preferably 55 to 90/1 to 40/0.01 to 10/0 to 5, further preferably 60 to 80/3 to 30/0.01 to 5/0 to 1, and particularly preferably 60 to 70/10 to 25/0.01 to 3/0 as expressed by mass % on the solid content basis. The content ratio among the respective components is selected from such various ranges so that the sum of the components is 100 mass %. The above content ratio allows performances such as sensitivity, resolution, and developability to be further excellent.

The method for preparing the composition for optical component formation of the present embodiment is not particularly limited, and examples thereof include a method including dissolving the respective components in the solvent in use to thereby provide a homogeneous solution, and thereafter, if necessary, filtering the solution by, for example, a filter having a pore size of about 0.2 μm.

The composition for optical component formation according the present embodiment can include resin as long as the objects of the present invention are not impaired. The type and the amount of the resin may be as disclosed in Patent Document 1.

The cured product of the present embodiment is obtained by curing of the composition for optical component formation, and can be used for various resins. The cured product can be used as a highly versatile material imparting various characteristics such as a high melting point, a high refractive index and high transparency in various applications. The cured product can be obtained by subjecting the composition to a known method depending on each compositional profile, such as light irradiation or heating.

The cured product can be used for various synthetic resins such as an epoxy resin, a polycarbonate resin and an acrylic resin, and furthermore optical components such as a lens and an optical sheet, by means of functionality.

### 6. Purification Method

The purification method of the polymer or compound is not particularly limited, and the method disclosed in International Publication No. 2015/080240 and the method disclosed in International Publication No. 2018/159707 may be used. More specifically, those purification methods include the step of dissolving the polymer or compound in an organic solvent which is always incompatible with water to give an organic phase, and bringing the organic phase into contact with an acidic aqueous solution to perform extraction treatment to transfer metal components in the organic phase including the polymer or compound and the organic solvent to the aqueous phase, and then separating the organic phase from the aqueous phase. The organic solvent which is always incompatible with water is an organic solvent generally classified into a water-insoluble solvent. The organic solvent is not particularly limited, and an organic solvent safely applicable to semiconductor manufacturing process is preferred. The organic solvent is usually used at a ratio of about 1 to 100 times the mass of the compound to be used.

Specific examples of the organic solvent include those disclosed in International Publication No. 2015/080240. Among them, toluene, 2-heptan, cyclohexanone, cyclopentanone, methyl isobutyl ketone, propylene glycol monomethyl ether acetate and ethyl acetate are preferred, and cyclohexanone and propylene glycol monomethyl ether acetate are particularly preferred.

The acidic aqueous solution is selected from aqueous solutions prepared by dissolving a commonly known organic or inorganic compound in water. Examples thereof include those disclosed in International Publication No. 2015/080240. Those acidic aqueous solutions may be used alone, respectively, or in combination of two or more. Examples of acidic aqueous solutions include an aqueous solution of mineral acid and an aqueous solution of organic acid. Examples of aqueous solutions of mineral acid include an aqueous solution including one or more selected from the group consisting of hydrochloric acid, sulfuric acid, nitric acid and phosphoric acid. Examples of aqueous solutions of organic acid include an aqueous solution including one or more selected from the group consisting of acetic acid, propionic acid, oxalic acid, malonic acid, succinic acid, fumaric acid, maleic acid, tartaric acid, citric acid, methanesulfonic acid, phenolsulfonic acid, p-toluenesulfonic acid and trifluoroacetic acid. The acidic aqueous solution has a pH in the range of about 0 to 5, and preferably about 0 to 3.

### EXAMPLES

### Example 1

### (1) Synthesis of BPAO

A 100 mL round bottom flask was charged with 20 mmol (4.56 g) of bisphenol A (BPA), 50 mmol (17.63 g) of cesium carbonate, 4.0 mmol (0.64 g) of KI and 20 mL of DMF, and the mixture was stirred for 1 hour at 80° C. Next, 30 mmol (3.61 g) of 3-(chloromethyl)-3-methyloxetane (CMO) was added thereto to perform the reaction at 70° C. for 8 hours, and the same amount of CMO was further added thereto, and the reaction was performed at 70° C. for 8 hours.

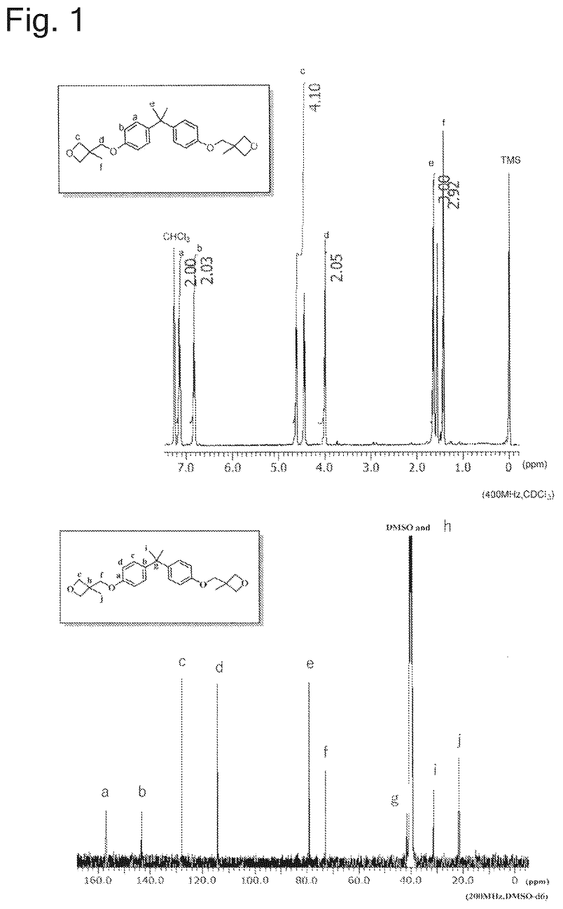

The reaction mixture was filtered and 1N hydrochloric acid was added to the filtrate to give a precipitate. Furthermore, the resultant was filtered using Kiriyama funnel to separate the precipitate by filtration. The precipitate was subjected to column separation using chloroform to isolate white solid as a target product (BPAO). The results are as follows. The ¹H-NMR spectrum and ¹³C-NMR spectrum are shown in FIG. 1.
Amount of yield; 7.179 g
Yield: 90%
Melting point: 161.0 to 163.0° C.

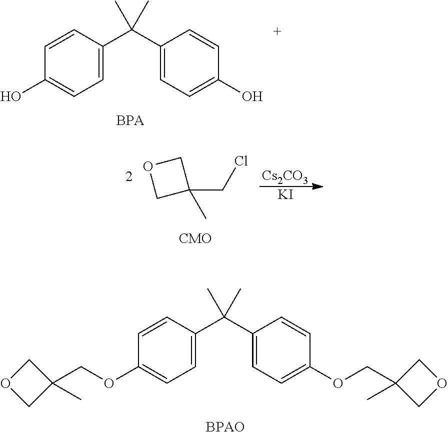

### (2) Synthesis of Poly(BPAO-Co-TeCl₄)

A flask was charged with BPAO synthesized as described above, TeCl₄ and catalyst to perform polymerization reaction. The conditions were as follows.
Solvent: chloroform
Concentration of reactant: 1M
Time: 6 hours
Temperature: room temperature
Feed ratio (molar ratio): described in Table 1

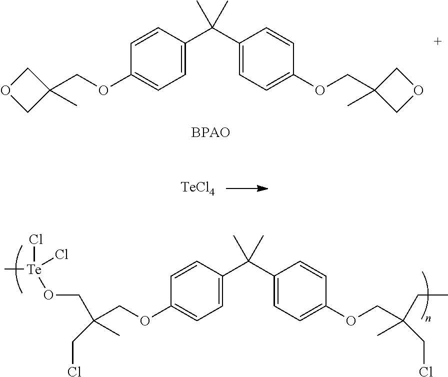

TABLE 1

| | feed ratio (BPAO/ | | | | | |
|---|---|---|---|---|---|---|
| | TeCl₄/AlCl₃/ | yeild | conversion | | M_{w}/ | |
| Run | BF₃O(C₂H₅)₂₎ | (%) | (%)¹⁾ | Mₙ²⁾ | Mₙ²⁾ | T_{d}^{5%3)} |
| 1 | 1/0.05/0/0 | 94 | 10 | 2,000 | 1.08 | — |
| 2 | 1/0.25/0/0 | 98 | 22 | 4,400 | 1.13 | — |
| 3 | 1/0.5/0/0 | 83 | 58 | 6,500 | 1.20 | — |
| 4 | 1/0.75/0/0 | 75 | 81 | 5,900 | 1.15 | — |
| 5 | 1/1/0/0 | 65 | 100 | 6,200 | 1.10 | 178 |
| 6 | 1/4/0/0 | 40 | 100 | 4,500 | 1.04 | — |
| 7 | 1/1/0.02/0 | 95 | 100 | 6,600 | 1.03 | — |
| 8 | 1/1/0/0.02 | 71 | 100 | 815,100 | 1.56 | — |

¹⁾By ¹H-NMR
²⁾By SEC (size exclusion chromatography)
³⁾By TGA (thermogravimetric analysis)

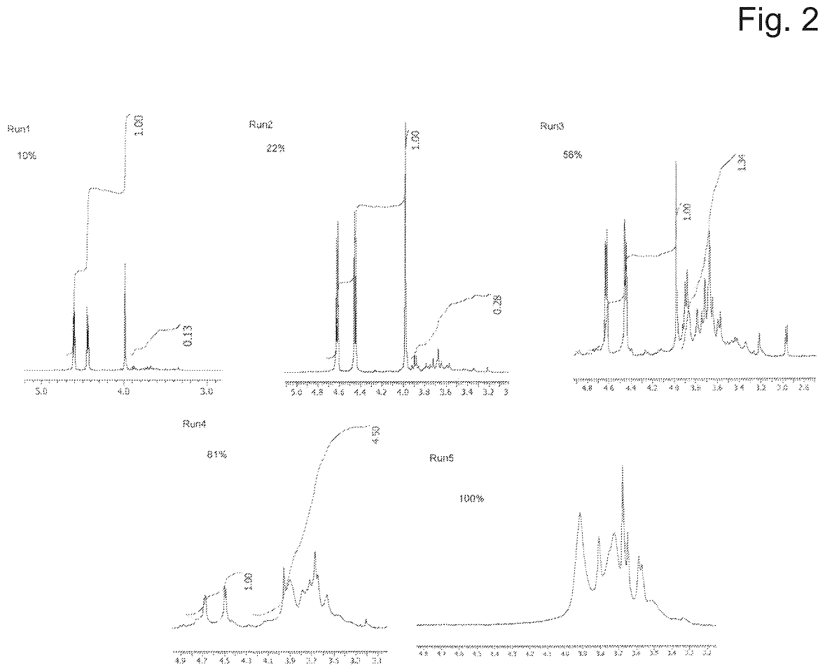

The results of the measurement of ¹H-NMR, in which each of the reaction solutions of Run 1 to Run 5 in Table 1 was directly added to deuterated chloroform, are shown in FIG. 2. In Run 1, since the conversion ratio of the raw materials was 10%, a peak of the raw material derived from 3-(chloromethyl)-3-methyloxetane (CMO) (4.4 to 4.7 ppm) was detected. Peaks derived from CMO reduced along with the improvement in the conversion ratio of the raw materials after Run 2, and no peak derived from CMO was observed in Run 5 (conversion ratio of raw material: 100%).

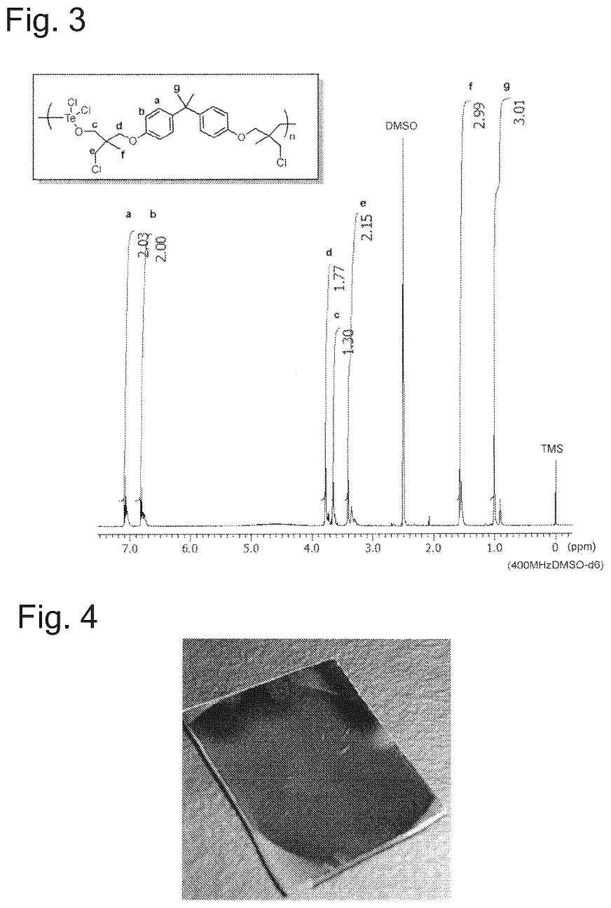

The resulting crude polymer was purified by re-precipitation using ether. The heat resistance of the polymer after purification was evaluated. The results are shown in Table 1. The heat resistance was evaluated using TGA (TGA-50/51 manufactured by Shimadzu Corporation, under nitrogen atmosphere, temperature-increasing rate 10° C./minute) at T_{d}^{5%} (5% weight loss temperature). The ¹H-NMR spectrum is shown in FIG. 3. Furthermore, 2 mg of the polymer was dissolved in 2 mL of each of the following solvents at 20° C., and conditions of the solutions were visually observed to perform a solubility test. The evaluation results are shown in Table 1-1. ++ means that the polymer was easily soluble and − means that the polymer was insoluble.

TABLE 1-1

| Solvent | Solubility |
|---|---|
| Acetone | ++ |
| Methanol | ++ |
| Ethanol | ++ |
| Ethyl acetate | ++ |
| Chloroform | +− |
| DCM | −− |
| MEK | ++ |
| THF | ++ |
| DMF | ++ |
| DMSO | ++ |
| Hexane | −− |
| Water | −− |

### Example 2

The polymer obtained in Run 5 of Example 1 was dissolved in THF to give a composition with a concentration of 3 mass %. The solution was spin-coated on a silicon wafer under the following conditions and post-baked to prepare a film. The solution had good film formability.
Number of rotations: 3,000 rpm
Time: 5 seconds
Post-baking: 90° C.

The resulting film has a film thickness of 100 nm and a refractive index of 1.60 as measured by an ellipsometer (measurement wavelength: 636 nm). A photograph of the film is shown in FIG. 4. The film formed as described above can be suitably used as a resist film, an underlayer film for lithography and an optical component.

### Example 3

A 30 mL round bottom flask was charged with 30 mmol (3.61 g) of 3-(chloromethyl)-3-methyloxetane (CMO), 15 mmol (4.41 g) of TeCl₄ and 10 mL of dichloromethane, and the mixture was stirred at room temperature for 3 hours to perform reaction. Next, the reaction solution was concentrated using an evaporator. Dichloromethane was added to the concentrate to dilute the concentrate, and this was injected into an excess amount of hexane to give a precipitate. This re-precipitation process was repeated three times. Next, the precipitate was filtered using a membrane filter to give a target compound (gray solid). The results are shown below.
Amount of yield; 0.741 g
Yield: 8%

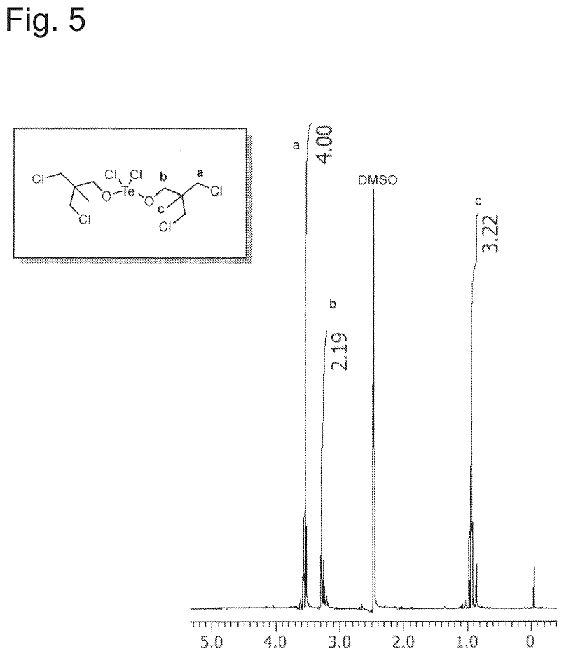

The reaction mixture was filtered and 1N hydrochloric acid was added to the filtrate to give a precipitate. Furthermore, the resultant was filtered using Kiriyama funnel to separate the precipitate by filtration. The precipitate was subjected to column separation using chloroform to isolate white solid as a target product. The results are as follows. The ¹H-NMR spectrum is shown in FIG. 5.
Amount of yield: 7.179 g
Yield: 90%
Melting point: 161.0 to 163.0° C.

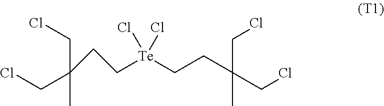

### Example 4

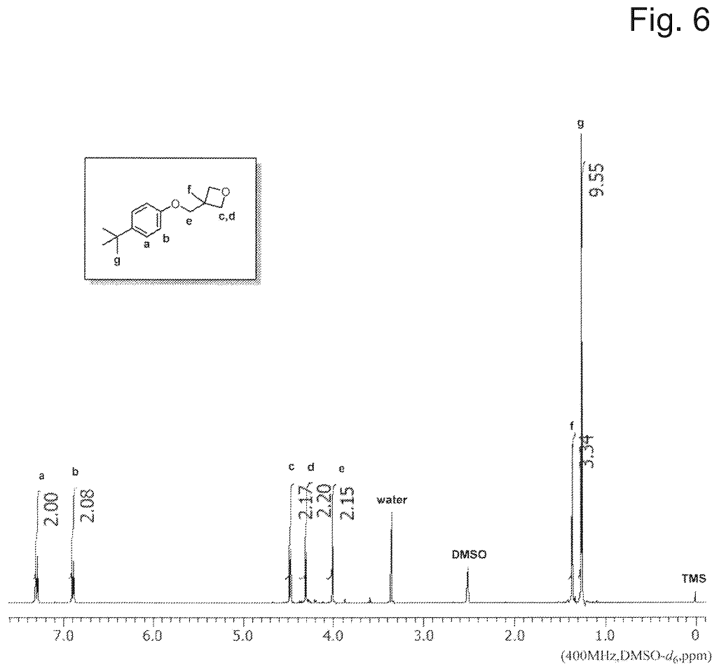

The following compound tBPO was synthesized by the same method as the method explained in “Synthesis of BPAO” except for using p-t-butylphenol instead of bisphenol A. The ¹H-NMR of tBPO is shown in FIG. 6.

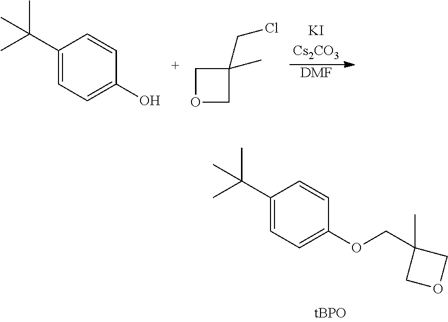

Subsequently, the following compound was obtained in the same manner as in Example 3, except for using tBPO instead of CMO.

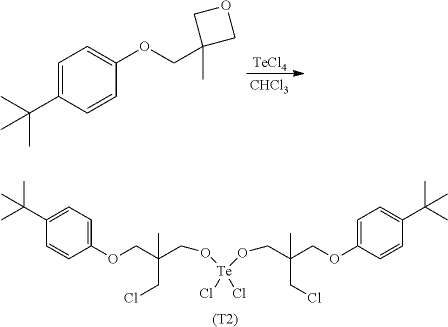

### [Example 5] Consideration of Feed Ratio for Poly(BPAE-Co-TeCl₂)

A 10 mL round bottom flask was charged with TeCl₄ in the amount shown in Table 2, and the content was subjected to purging with nitrogen. The feed ratios in Table 2 are in the molar ratio. Next, 1.0 mmol (0.340 g) of bisphenol A glycidyl ether (denoted as BPAE) dissolved in 2.0 mL of chloroform was gradually added to the flask using a syringe. The mixture was stirred at room temperature for 5 hours to perform the reaction, and then was filtered and the filtrate was concentrated. The results of analysis are shown in Table 2. The conversion ratio was determined by quantifying the epoxy groups by ¹H-NMR. The molecular weight and the molecular weight distribution were determined by SEC. T_{d}^{5%} (5% weight loss temperature) was determined using TGA (TGA-50/51 manufactured by Shimadzu Corporation, under nitrogen atmosphere, temperature-increasing rate 10° C./minute).

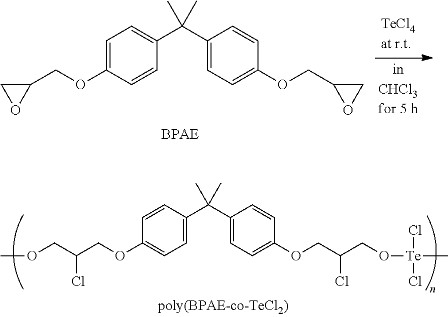

TABLE 2

| | Feed ratio | Conv. | Mₙ | T_{d}^{5% c)} |
|---|---|---|---|---|
| Run | (BPAE/TeCl₄) | (%) ^{a)} | (M_{w}/Mₙ) ^{b)} | (° C.) |
| 1 | 1.00/0.05 | 11 | 1,900 (1.05) | — ^{d)} |
| 2 | 1.00/0.25 | 20 | 2,400 (1.11) | — ^{d)} |
| 3 | 1.00/0.50 | 50 | 2,100 (1.14) | — ^{d)} |
| 4 | 1.00/0.75 | 78 | 2,200 (1.13) | — ^{d)} |
| 5 | 1.00/1.00 | 100 | 2,600 (1.21) | — ^{d)} |
| 6 | 1.00/4.00 | 100 | 2,000 (1.11) | — ^{d)} |

^{a)} Calculated by quantification of epoxy group by ¹H-NMR
^{b)} By SEC
^{c)} By TGA
^{d)} Not measured

The conversion ratio was found to increase as the amount of charge of TeCl₄ is increased, and a conversion ratio of 100% was found to be achieved at a feed ratio of 1:1. This shows that TeCl₄ reacted catalytically and the reaction ratio of the epoxide, BPAE, to TeCl₄ was 1:1. The viscous polymer solution after the completion of the reaction suggested that a polymer was obtained, but the results of size exclusion chromatography did not confirm the polymer. The instability of the Te—O bond is a possible cause. TGA of the precipitate produced when the polymer was dissolved in the solvent for SEC measurements was also performed. As a result, a TGA curve similar to that of an inorganic product was obtained. This also suggests that inorganic tellurium was formed due to decomposition in the measurement solvent.

### [Example 6] Consideration of Feed Ratio and Solvent for Poly(MTPEP-Co-TeCl₂)

TeCl₄ was introduced into a test tube in the amount shown in Table 3, and the content was subjected to purging with nitrogen. The feed ratios in Table 3 are in the molar ratio. Next, 1.0 mmol (0.460 g) of 4,4′,4″-trihydroxytriphenylmethane triglycidyl ether (denoted as MTPEP) dissolved in 2.0 mL of chloroform or methyl ethyl ketone (MEK) was gradually added to the test tube using a syringe, and the mixture was reacted at room temperature for 24 hours. Then the resulting solution was added dropwise to ethanol to give orange viscous liquid. ¹H-NMR results showed the disappearance of peaks derived from the epoxide of MTPEP, confirming the progress of the reaction. The results of analysis are shown in Table 3. The analysis was performed by the method described in Example 5.

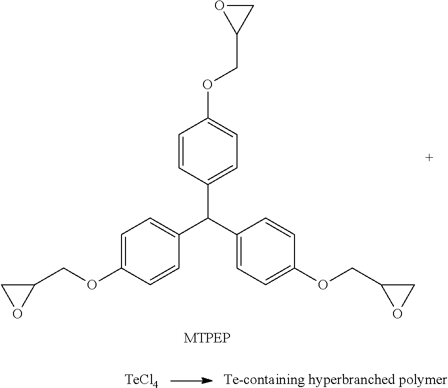 [SMILES: c1cc(C(c2ccc(OCC3CO3)cc2)c2ccc(OCC3CO3)cc2)ccc1OCC1CO1 ; "TeCl4" ; "Te-containing hyperbranched polymer"]

TABLE 3

| | | Feed ratio | Mₙ | T_{d}^{5% c)} | |
|---|---|---|---|---|---|
| Run | solvent | (MTPEP/TeCl₄) | (M_{w}/Mₙ) ^{b)} | (° C.) | n_{D}^{)d} |
| 1 | CHCl₃ | 1.00/0.75 | 2,800 (1.11) | — ^{f)} | — ^{f)} |
| 2 | | 1.00/1.00 | 2,800 (1.34) | — ^{f)} | — ^{f)} |
| 3 | | 1.00/1.50 | 3,600 (1.23) | — ^{f)} | — ^{f)} |
| 4 | | 1.00/2.00 | — ^{e)} | — ^{f)} | — ^{f)} |
| 5 | | 1.00/2.50 | — ^{e)} | — ^{f)} | — ^{f)} |
| 6 | MEK | 1.00/0.75 | 2,600 (1.15) | — ^{f)} | — ^{f)} |
| 7 | | 1.00/1.00 | 3,100 (1.21) | — ^{f)} | — ^{f)} |
| 8 | | 1.00/1.50 | 3,400 (1.14) | — ^{f)} | — ^{f)} |
| 9 | | 1.00/2.00 | — ^{e)} | — ^{f)} | — ^{f)} |
| 10 | | 1.00/2.50 | — ^{e)} | — ^{f)} | — ^{f)} |

^{b)} By SEC (solvent: DMF in which LiBr and H₃PO₄ dissolved, in terms of polystyrene)
^{c)} By TGA
^{)d}By ellipsometer
^{e)} Not measurable by SEC
^{f)} Not measured

d) By ellipsometer
e) Not measurable by SEC
f) Not measured

The molecular weight of the resulting compounds was determined using SEC, and the results confirmed that a polymer compound with a molecular weight of 2,600 to 3,600 was obtained. However, at a feed ratio of 1.00/2.00 to 1.2.50, the measurement by SEC was impossible.

### [Example 7] Consideration of Feed Ratio and Solvent for Poly(MTPOX-Co-TeCl₂)

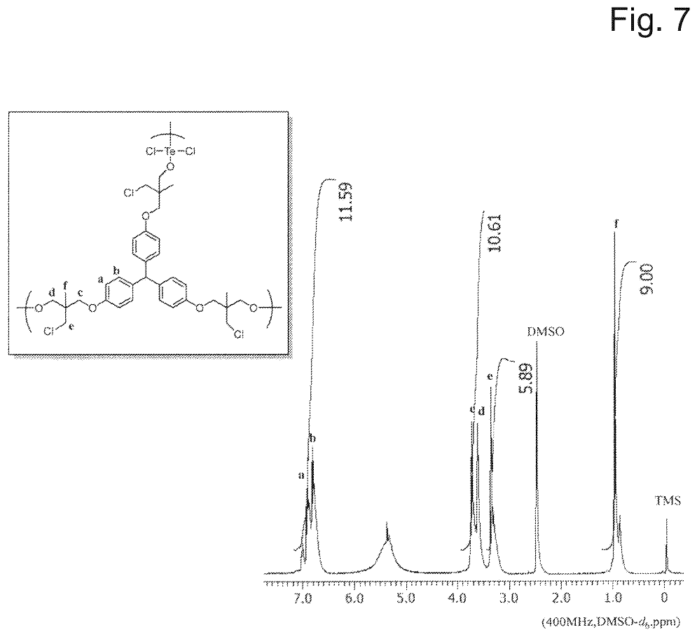

TeCl₄ was introduced into a test tube in the amount shown in Table 4, and the content was subjected to purging with nitrogen. The feed ratios in Table 4 are in the molar ratio. Next, 1.0 mmol (0.544 g) of MTPOX dissolved in 2.0 mL of chloroform or MEK was gradually added to the test tube using a syringe, and the mixture was reacted at room temperature for 24 hours. Then the resulting solution was added dropwise to ethanol to give orange solid. The ¹H-NMR chart is shown in FIG. 7 and the results of analysis are shown in Table 4. The molecular weight, the molecular weight distribution and T_{d}^{5%} were determined as described above. The refractive index (np) was measured by the method described in Example 2.

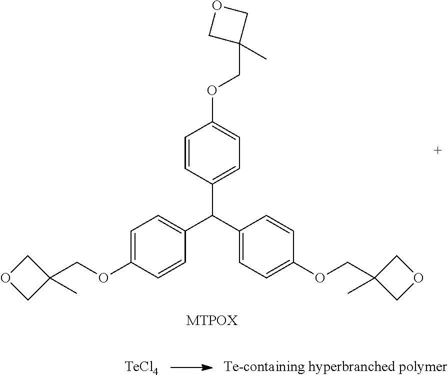 [SMILES: CC1(COc2ccc(C(c3ccc(OCC4(C)COC4)cc3)c3ccc(OCC4(C)COC4)cc3)cc2)COC1 ; "TeCl4" ; "Te-containing hyperbranched polymer"]

TABLE 4

| | | Feed ratio | Mₙ | | T_{d}^{5% c)} | |
|---|---|---|---|---|---|---|
| Run | solvent | (MTPOX/TeCl₄) | (M_{w}/Mₙ) ^{b)} | | (° C.) | n_{D}^{)d} |
| 1 | CHCl₃ | 1.00/0.75 | 6,400 | (1.69) | 280 | 1.58 |
| 2 | | 1.00/1.00 | 6,400 | (1.20) | 281 | 1.58 |
| 3 | | 1.00/1.50 | 6,300 | (1.37) | 287 | 1.59 |
| 4 | | 1.00/2.00 | 6,600 | (2.34) | — ^{f)} | — ^{f)} |
| 5 | | 1.00/2.50 | 6,600 | (2.36) | — ^{f)} | — ^{f)} |
| 6 | MEK | 1.00/0.75 | 14,700 | (5.11) | 340 | — ^{f)} |
| 7 | | 1.00/1.00 | 15,600 | (10.1) | 326 | — ^{f)} |
| 8 | | 1.00/1.50 | 19,400 | (9.71) | 348 | 1.58 |

| | | | | |
|---|---|---|---|---|
| 9 | 1.00/2.00 | — ^{e)} | 318 | — ^{f)} |
| 10 | 1.00/2.50 | — ^{e)} | 344 | — ^{f)} |

^{b)} By SEC (solvent: DMF in which LiBr and H₃PO₄ dissolved, in terms of polystyrene)
^{c)} By TGA
^{)d}By ellipsometer
^{e)} Not measurable by SEC
^{f)} Not measured

The measurement of the molecular weight by SEC revealed that a polymer with a higher molecular weight was obtained by using MEK as the solvent. However, since the polymer has a very wide molecular weight distribution, the polymer is less suitable for resist materials. In addition, if TeCl₂ is present at the ends, the polymer is likely to be more sensitive to moisture. From the above, chloroform was selected as the solvent considering use in resist materials, and resist properties of the polymer synthesized in Run 2 were evaluated below.

Film forming was performed in the same manner as in Example 2 except that the polymer synthesized in Run 2 was dissolved in THE to give a composition with a concentration of 2 mass %.
Solvent: THF
Concentration: 2 mass %

The resulting film was evaluated for the refractive index and film thickness in the same manner as in Example 2. The results are shown below.
Refractive index: 1.584
Film thickness: 60 nm

The results of evaluation of the solubility of the polymer synthesized in Run 2 are shown below. The solubility was evaluated in the same manner as in Example 1. The results are shown in Table 5.

TABLE 5

| Solvent | Solubility |
|---|---|
| Chloroform | ++ |
| Dichloromethane (DCM) | +− |
| Methyl ethyl ketone (MEK) | ++ |
| Tetrahydrofuran (THF) | ++ |
| Propylene glycol monomethyl ether (PGME) | ++ |
| Propylene glycol monomethyl ether acetate (PGMEA) | ++ |
| Acetonitrile | +− |
| Hexane | +− |
| Toluene | −− |
| Methanol | +− |
| Ethyl acetate | ++ |
| Diethyl ether | ++ |
| Tetramethylammonium hydroxide aqueous solution | −− |
| (TMAHaq) | |

### Synthetic Comparative Example 1

A four-neck flask with an internal volume of 10 L equipped with a Dimroth condenser, a thermometer and a stirring blade, which can be bottomed out, was prepared. The four-neck flask was charged with 1.09 kg of 1,5-dimethylnaphthalene (7 mol, manufactured by Mitsubishi Gas Chemical Company, Inc.), 2.1 kg of a 40 mass % formalin aqueous solution (28 mol in terms of formaldehyde, manufactured by Mitsubishi Gas Chemical Company, Inc.) and 0.97 mL of 98 mass % sulfuric acid (manufacture by KANTO CHEMICAL CO., INC.) under nitrogen atmosphere. The mixture was reacted at normal pressure for 7 hours under reflux at 100° C. Then 1.8 kg of ethylbenzene (special grade, made by Wako Pure Chemical Industries, Ltd.) was added to the reaction solution as a dilution solvent, and after the solution was allowed to stand, the lower aqueous phase was removed. After further neutralization and washing, ethylbenzene and unreacted 1,5-dimethylnaphthalene were distilled off under reduced pressure to obtain 1.25 kg of dimethylnaphthalene formaldehyde resin, which was a light brown solid. The resulting dimethylnaphthalene formaldehyde had a molecular weight Mn of 562.

Subsequently, a four-neck flask with an internal volume of 0.5 L equipped with a Dimroth condenser, a thermometer and a stirring blade was prepared. The four-neck flask was charged with 100 g (0.51 mol) of the above dimethylnaphthalene formaldehyde resin and 0.05 g of p-toluenesulfonic acid under nitrogen atmosphere. The temperature was increased to 190° C. and the mixture was heated for 2 hours, and then stirred. Then 52.0 g (0.36 mol) of 1-naphthol was also added thereto and the temperature was increased to 220° C. and the mixture was reacted for 2 hours. After dilution with solvent, the resultant was neutralized and rinsed with water, and the solvent was removed under reduced pressure to give 126.1 g of a black-brown solid modified resin (CR-1). The resulting resin (CR-1) had Mn: 885, Mw: 2220 and Mw/Mn: 4.17. Mn, Mw and Mw/Mn of resin (CR-1) were determined by gel permeation chromatography (GPC) analysis under the following measurement conditions in terms of polystyrene.
Apparatus: Shodex GPC-101 Model (manufactured by SHOWA DENKO K.K.)
Column: KF-80M×3
Eluent: THF 1 mL/minute
Temperature: 40° C.

### (Synthesis Example 1) Synthesis of AC-1

4.15 g of 2-methyl-2-methacryloyloxyadamantane, 3.00 g of methacryloyloxy-y-butyrolactone, 2.08 g of 3-hydroxy-1-adamantyl methacrylate and 0.38 g of azobisisobutyronitrile were dissolved in 80 mL of tetrahydrofuran to prepare a reaction solution. The temperature was kept at 63° C. with stirring the reaction solution under nitrogen atmosphere to perform polymerization for 22 hours. Then the reaction solution was added dropwise to 400 mL of n-hexane. The resulting resin was coagulated and purified, and the white powder produced was filtered and then dried under reduced pressure at 40° C. overnight to give AC-1 represented by the following formula.

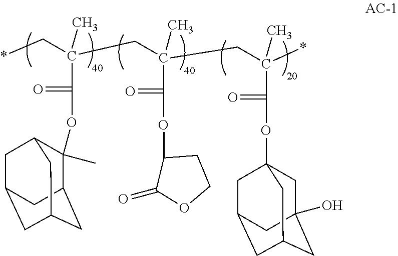

In formula AC-1, “40”, “40”, “20” represent the ratio of the respective constituent units, and do not mean that the polymer is a block copolymer.

### Example A, Comparative Example A

Compositions for lithography film formation (compositions for resist film formation) with the formulation shown in Table 6 were individually prepared using the following compound or polymer.
Poly(BPAO-co-TeCl₄), resin obtained in Example 1
Compound (T1) obtained in Example 3
Compound (T2) obtained in Example 4
Poly(BPAE-co-TeCl₂) obtained in Example 5
Poly(MTPEP-co-TeCl₂) obtained in Example 6
Poly(MTPOX-co-TeCl₂) obtained in Example 7
CR-1

The following materials were used as the acid generating agent, the acid diffusion controlling agent, the acid crosslinking agent or the organic solvent. In Table 6, values in parenthesis are the amount mixed (part(s) by mass).

### <Acid Generating Agent>

Triphenylsulfonium trifluoromethanesulfonate (TPS-109 (tradename)) manufactured by Midori Kagaku Co., Ltd.

Di-tert-butyl diphenyl iodonium nonafluorobutanesulfonate (DTDPI) manufactured by Midori Kagaku Co., Ltd.

Pyridinium p-toluenesulfonic acid manufactured by Kanto Chemical Co., Inc. (denoted as “PPTS” in tables)

### <Acid Diffusion Controlling Agent>

Tri-n-octylamine (TOA, manufactured by Kanto Chemical Co., Inc.)

### <Acid Crosslinking Agent>

NIKALAC MW-100 LM (MW-100 LM, tradename, manufactured by Sanwa Chemical Co., Ltd.)

NIKALAC MX270 manufactured by Sanwa Chemical Co., Ltd.

TMOM-BP manufactured by Honshu Chemical Industry Co., Ltd.

### <Organic Solvent>

Propylene glycol monomethyl ether (PGME manufactured by Kanto Chemical Co., Inc.)

TABLE 6

| | | | Acid | | |
|---|---|---|---|---|---|
| | | Acid | diffusion | Cross- | |
| | Compound | generating | controlling | linking | Organic |
| | or resin | agent | agent | agent | solvent |
| | (part(s) | (part(s) | (part(s) | (part(s) | (part(s) |
| | by mass) | by mass) | by mass) | by mass) | by mass) |
| Example | Product obtained | TPS-109 | TOA | MW-100LM | PGME |
| A1 | in Example 1 | (1) | (0.1) | (1) | (100) |
| | (5) | | | | |
| Example | Product obtained | TPS-109 | TOA | MW-100LM | PGME |
| A2 | in Example 1 | (1) | (0.1) | (1) | (100) |
| | (3) | | | | |
| Example | Product obtained | DTDPI | TOA | MW-100LM | PGME |
| A3 | in Example 1 | (1) | (0.1) | (1) | (100) |
| | (5) | | | | |
| Example | Product obtained | PPTS | TOA | MW-100LM | PGME |
| A4 | in Example 1 | (1) | (0.1) | (1) | (100) |
| | (5) | | | | |
| Example | Product obtained | TPS-109 | TOA | NIKALAC | PGME |
| A5 | in Example 1 | (1) | (0.1) | MX270 | (100) |
| | (5) | | | (1) | |
| Example | Product obtained | TPS-109 | TOA | TMOM-BP | PGME |
| A6 | in Example 1 | (1) | (0.1) | (1) | (100) |
| | (5) | | | | |
| Example | Product obtained | TPS-109 | TOA | MW-100LM | PGME |
| A7 | in Example 3 | (1) | (0.1) | (1) | (100) |
| | (5) | | | | |
| Example | Product obtained | TPS-109 | TOA | MW-100LM | PGME |
| A8 | in Example 4 | (1) | (0.1) | (1) | (100) |
| | (5) | | | | |
| Example | Product obtained | TPS-109 | TOA | MW-100LM | PGME |
| A9 | in Example 5 | (1) | (0.1) | (1) | (100) |
| | (5) | | | | |
| Example | Product obtained | DTDPI | TOA | MW-100LM | PGME |
| A10 | in Example 5 | (1) | (0.1) | (1) | (100) |
| | (5) | | | | |
| Example | Product obtained | PPTS | TOA | MW-100LM | PGME |
| A11 | in Example 5 | (1) | (0.1) | (1) | (100) |
| | (5) | | | | |
| Example | Product obtained | TPS-109 | TOA | NIKALAC | PGME |
| A12 | in Example 5 | (1) | (0.1) | MX270 | (100) |
| | (5) | | | (1) | |
| Example | Product obtained | TPS-109 | TOA | TMOM-BP | PGME |
| A13 | in Example 5 | (1) | (0.1) | (1) | (100) |
| | (5) | | | | |
| Example | Product obtained | TPS-109 | TOA | MW-100LM | PGME |
| A14 | in Example 6 | (1) | (0.1) | (1) | (100) |
| | (5) | | | | |
| Example | Product obtained | TPS-109 | TOA | MW-100LM | PGME |
| A15 | in Example 7 | (1) | (0.1) | (1) | (100) |
| | (5) | | | | |
| Example | Product obtained | TPS-109 | TOA | MW-100LM | PGME |
| A16 | in Example 7 | (1) | (0.1) | (1) | (100) |
| | (3) | | | | |
| Example | Product obtained | DTDPI | TOA | MW-100LM | PGME |
| A17 | in Example 7 | (1) | (0.1) | (1) | (100) |
| | (5) | | | | |
| Example | Product obtained | PPTS | TOA | MW-100LM | PGME |
| A18 | in Example 7 | (1) | (0.1) | (1) | (100) |
| | (5) | | | | |
| Example | Product obtained | TPS-109 | TOA | NIKALAC | PGME |
| A19 | in Example 7 | (1) | (0.1) | MX270 | (100) |
| | (5) | | | (1) | |
| Example | Product obtained | TPS-109 | TOA | TMOM-BP | PGME |
| A20 | in Example 7 | (1) | (0.1) | (1) | (100) |
| | (5) | | | | |
| Comparative | CR-1 | TPS-109 | TOA | MW-100LM | PGME |
| Example A1 | (5) | (1) | (0.1) | (1) | (100) |

### EVALUATION METHOD

(1) Thin Film Formation with Composition for Resist Film Formation

After preparing the respective compositions for resist film formation according to the formulation described in Table 6, each of the compositions for resist film formation in a homogeneous state was applied to a clean silicon wafer by spin-coating. Then each composition was pre-baked (PB) in an oven at 110° C. to form a resist film with a thickness of 40 nm. The resist films were rated as G when a good thin film was formed, and rated as N when the formed film had defects.

### (2) Resist Pattern

Each of the resist films obtained in (1) above was irradiated with electron beam using an electron beam lithography system (ELS-7500 manufactured by ELIONIX INC., 50 keV) with a 1:1 line and space setting at intervals of 50 nm. After irradiation, the resist films were heated at 110° C. for 90 seconds, respectively, and developed by immersion in propylene glycol monomethyl ether (PGME) developing solution for 60 seconds. Each resist film was then rinsed with ultrapure water for 30 seconds and dried to form a resist pattern.

The shape of the resulting resist pattern of L/S (1:1) at intervals of 50 nm was observed using an electron microscope (S-4800, tradename) manufactured by Hitachi, Ltd. The shape of the resist pattern after development was evaluated based on the following criteria.
A: No pattern collapse and rectangularity is better than that in Comparative Example A1.
B: Rectangularity is better than that in Comparative Example A1, but one or two pattern collapses are observed within the area of 1 μm×1.5 μm.
B: Three or more pattern collapses are observed within the area of 1 μm×1.5 μm.
C: Equivalent to or inferior to Comparative Example A1

In Comparative Example A1, pattern collapses were observed in the resist pattern shape after development, and rectangularity was poor.

### (3) Etching Resistance

An etching test was performed on each of the resist films obtained in the above evaluation method (1), and the etching rate at that stage was measured. A comparative composition was also prepared using a novolac resin (PSM4357 (model number) from Gun Ei Chemical Industry Co.) instead of poly(BPAO-co-TeCl₄) in Example A1 in the formulation shown in Table 6. A resist film was prepared in the same manner as described in (1) above using the comparative composition, and the etching test was also performed on this resist film. Etching conditions are as follows.

### (Etching Conditions)

Etching apparatus: RIE-10NR (tradename) manufactured by Samco Inc.
Output: 50 W
Pressure: 20 Pa
Time: 2 minutes
Etching gas: Ar gas flow rate: CF₄ gas flow rate: O₂ gas flow rate=50:5:5 (sccm)

The etching resistance of each resist film was evaluated based on the etching rate of resist film obtained from the comparative composition using novolac resin, using the following evaluation criteria.

### Evaluation Criteria

A: The etching rate is less than-15% of that of the novolac resin resist film.
B: The etching rate is-15% to +5% of that of the novolac resin resist film.
C: The etching rate is more than +5% of that of the novolac resin resist film.

The evaluation results are shown in Table 7.

TABLE 7

Evaluation result

| | Thin film | Resist | Etching |
|---|---|---|---|
| | formation | pattern | resistance |
| Example A1 | G | B | A |
| Example A2 | G | B | A |
| Example A3 | G | B | A |
| Example A4 | G | B | A |
| Example A5 | G | B | A |
| Example A6 | G | B | A |
| Example A7 | G | B | A |
| Example A8 | G | B | A |
| Example A9 | G | A | B |
| Example A10 | G | A | B |
| Example A11 | G | A | B |
| Example A12 | G | A | B |
| Example A13 | G | A | B |
| Example A14 | G | A | B |
| Example A15 | G | B | A |
| Example A16 | G | B | A |
| Example A17 | G | B | A |
| Example A18 | G | B | A |
| Example A19 | G | B | A |
| Example A20 | G | B | A |
| Comparative | G | C | C |
| Example A1 | | | |

It is clear that the composition of Example A can form excellent resist patterns and films with excellent etching resistance.

### Example B, Comparative Example B

### (1) Formation of Resist Underlayer Film

Compositions for resist underlayer film formation with the formulation shown in Table 8 were individually prepared using the same Te-containing compound or the same Te-containing polymer as those in Example A. Next, each of the compositions for resist underlayer film formation was spin-coated on a silicon substrate, and then baked at 240° C. for 60 seconds and additionally baked at 400° C. for 120 seconds to give resist underlayer films with a film thickness of 70 nm. The following materials were used as the acid generating agent, the acid crosslinking agent and the organic solvent.

### <Acid Generating Agent>

Di-tert-butyl diphenyl iodonium nonafluoromethanesulfonate (denoted as “DTDPI” in tables) (manufactured by Midori Kagaku Co., Ltd.)

Triphenylsulfonium trifluoromethanesulfonate (TPS-109 (tradename)) (manufactured by Midori Kagaku Co., Ltd.)

Pyridinium p-toluenesulfonic acid manufactured by Kanto Chemical Co., Inc. (denoted as “PPTS” in tables)

### <Acid Crosslinking Agent>

“NIKALAC MW270 (denoted as “MW270” in tables) (product name, manufactured by Sanwa Chemical Co., Ltd.)

“NIKALAC MW-100 LM” (denoted as “MW-100 LM” in tables) (tradename, manufactured by Sanwa Chemical Co., Ltd.)

TMOM-BP manufactured by Honshu Chemical Industry Co., Ltd.

### <Organic Solvent>

Propylene glycol monomethyl ether (also referred to as “PGME” in tables)

TABLE 8

| | | Acid | Cross- | |
|---|---|---|---|---|
| | Compound | generating | linking | Organic |
| | or resin | agent | agent | solvent |
| | (part(s) | (part(s) | (part(s) | (part(s) |
| | by mass) | by mass) | by mass) | by mass) |
| Example B1 | Product obtained | DTDPI | MX270 | PGME |
| | in Example 1 | (0.5) | (0.5) | (90) |
| | (10) | | | |
| Example B2 | Product obtained | DTDPI | MX270 | PGME |
| | in Example 1 | (0.5) | (0.5) | (90) |
| | (8) | | | |
| Example B3 | Product obtained | TPS-109 | MX270 | PGME |
| | in Example 1 | (0.5) | (0.5) | (90) |
| | (10) | | | |
| Example B4 | Product obtained | PPTS | MX270 | PGME |
| | in Example 1 | (0.5) | (0.5) | (90) |
| | (10) | | | |
| Example B5 | Product obtained | DTDPI | MW-100LM | PGME |
| | in Example 1 | (0.5) | (0.5) | (90) |
| | (10) | | | |
| Example B6 | Product obtained | DTDPI | TMOM-BP | PGME |
| | in Example 1 | (0.5) | (0.5) | (90) |
| | (10) | | | |
| Example B7 | Product obtained | N/A | N/A | PGME |
| | in Example 1 | | | (90) |
| | (10) | | | |
| Example B8 | Product obtained | DTDPI | MX270 | PGME |
| | in Example 3 | (0.5) | (0.5) | (90) |
| | (10) | | | |
| Example B9 | Product obtained | DTDPI | MX270 | PGME |
| | in Example 4 | (0.5) | (0.5) | (90) |
| | (10) | | | |
| Example B10 | Product obtained | DTDPI | MX270 | PGME |
| | in Example 5 | (0.5) | (0.5) | (90) |
| | (10) | | | |
| Example B11 | Product obtained | DTDPI | MX270 | PGME |
| | in Example 5 | (0.5) | (0.5) | (90) |
| | (8) | | | |
| Example B12 | Product obtained | TPS-109 | MX270 | PGME |
| | in Example 5 | (0.5) | (0.5) | (90) |
| | (10) | | | |
| Example B13 | Product obtained | PPTS | MX270 | PGME |
| | in Example 5 | (0.5) | (0.5) | (90) |
| | (10) | | | |
| Example B14 | Product obtained | DTDPI | MW-100LM | PGME |
| | in Example 5 | (0.5) | (0.5) | (90) |
| | (10) | | | |
| Example B15 | Product obtained | DTDPI | TMOM-BP | PGME |
| | in Example 5 | (0.5) | (0.5) | (90) |
| | (10) | | | |
| Example B16 | Product obtained | N/A | N/A | PGME |
| | in Example 5 | | | (90) |
| | (10) | | | |
| Example B17 | Product obtained | DTDPI | MX270 | PGME |
| | in Example 6 | (0.5) | (0.5) | (90) |
| | (10) | | | |
| Example B18 | Product obtained | DTDPI | MX270 | PGME |
| | in Example 7 | (0.5) | (0.5) | (90) |
| | (10) | | | |
| Example B19 | Product obtained | DTDPI | MX270 | PGME |
| | in Example 7 | (0.5) | (0.5) | (90) |
| | (8) | | | |
| Example B20 | Product obtained | TPS-109 | MX270 | PGME |
| | in Example 7 | (0.5) | (0.5) | (90) |
| | (10) | | | |
| Example B21 | Product obtained | PPTS | MX270 | PGME |
| | in Example 7 | (0.5) | (0.5) | (90) |
| | (10) | | | |
| Example B22 | Product obtained | DTDPI | MW-100LM | PGME |
| | in Example 7 | (0.5) | (0.5) | (90) |
| | (10) | | | |
| Example B23 | Product obtained | DTDPI | TMOM-BP | PGME |
| | in Example 7 | (0.5) | (0.5) | (90) |
| | (10) | | | |
| Example B24 | Product obtained | N/A | N/A | PGME |
| | in Example 7 | | | (90) |
| | (10) | | | |

### (2) Formation and Evaluation of Resist Film

A resist solution for ArF was applied to each of the resist under layer films formed in (1) above and this was baked at 130° C. for 60 seconds to give a photoresist film with a film thickness of 140 nm. The resist solution for ArF was prepared by mixing 5 parts by mass of resin (AC-1) prepared in Synthetic Example 1 above, 1 part by mass of triphenylsulfonium nonafluoromethanesulfonate, 2 parts by mass of tributylamine and 92 parts by mass of PGMEA.

Next the photoresist film was exposed by using electron beam lithography system “ELS-7500” (product name, manufactured by ELIONIX INC., 50 keV). Then, the film was baked at 115° C. for 90 seconds (PEB), and developed using a 2.38 mass % tetramethylammonium hydroxide (TMAH) aqueous solution for 60 seconds to give a positive resist pattern.

The results of the observation of defects in the 55 nm L/S (1:1) and 80 nm L/S (1:1) resist patterns obtained are shown in Table 9. In the table, the result “good” for the “resist pattern after development” indicates that no pattern collapse was observed in the formed resist pattern, while “poor” indicates that pattern collapse was observed in the formed resist pattern. In the observation results, the smallest line width with no pattern collapse and good rectangularity was defined as “resolution performance” and used as an evaluation index. Furthermore, the minimum amount of electron beam energy with which good pattern shape could be drawn was defined as the “sensitivity” and used as an evaluation index. The results are shown in Table 9.

### Comparative Example B1

A positive resist pattern was obtained in the same manner as in Example B1 except for forming a photoresist film directly on a SiO₂ substrate without forming the underlayer film. The results are shown in Table 9.

TABLE 9

| | Composition | | | Resist |
|---|---|---|---|---|
| | for resist | Resolution | Sensi- | pattern |
| | underlayer | performance | tivity | after |
| | film formation | (nmL/S) | (μC/cm²) | development |
| Example B1 | Example B1 | 55 | 10 | Good |
| Example B2 | Example B2 | 55 | 10 | Good |
| Example B3 | Example B3 | 55 | 10 | Good |
| Example B4 | Example B4 | 55 | 10 | Good |
| Example B5 | Example B5 | 55 | 10 | Good |
| Example B6 | Example B6 | 55 | 10 | Good |
| Example B7 | Example B7 | 55 | 10 | Good |
| Example B8 | Example B8 | 55 | 10 | Good |
| Example B9 | Example B9 | 55 | 10 | Good |
| Example B10 | Example B10 | 55 | 10 | Good |
| Example B11 | Example B11 | 55 | 10 | Good |
| Example B12 | Example B12 | 55 | 10 | Good |
| Example B13 | Example B13 | 55 | 10 | Good |
| Example B14 | Example B14 | 55 | 10 | Good |
| Example B15 | Example B15 | 55 | 10 | Good |
| Example B16 | Example B16 | 55 | 10 | Good |
| Example B17 | Example B17 | 55 | 10 | Good |
| Example B18 | Example B18 | 55 | 10 | Good |
| Example B19 | Example B19 | 55 | 10 | Good |
| Example B20 | Example B20 | 55 | 10 | Good |
| Example B21 | Example B21 | 55 | 10 | Good |
| Example B22 | Example B22 | 55 | 10 | Good |
| Example B23 | Example B23 | 55 | 10 | Good |
| Example B24 | Example B24 | 55 | 10 | Good |
| Comparative | N/A | 80 | 26 | Poor |
| Example B1 | | | | |

It is clear that the composition of Example B can form highly sensitive films.

1. A polymer comprising tellurium in the main chain, which is prepared by reacting:
(A) a compound having a plurality of cyclic (thio) ether groups, and
(B) TeX₄, wherein X is a halogen atom.

2. The polymer according to claim 1, wherein (A) is a compound represented by formula (A1): 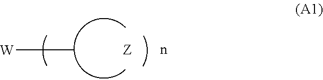
wherein n is 2 to 4,
W is an organic group having 10 to 30 carbon atoms,
the cyclic structure including Z is a group having a cyclic ether group or a cyclic thioether group, and
Z is O or S.

3. The polymer according to claim 2, wherein
W is a group represented by —Z—Ar—Z—, and
Ar is an organic group having an aromatic ring or a heterocyclic ring.

4. The polymer according to claim 3, wherein
Ar is a group represented by —Ar′—U—Ar′—
Ar′ is an aromatic group or a heterocyclic aromatic group, and
U is a divalent organic group or a single bond.

5. The polymer according to claim 1, wherein the cyclic (thio) ether group of (A) has a 3 to 5-membered ring structure.

6. A method for producing the polymer according to claim 1, comprising a step of subjecting (A) and (B) to an addition reaction.

7. The method according to claim 6, wherein the reaction is performed in the presence of Lewis acid.

8. A composition comprising the polymer according to claim 1.

9. The composition according to claim 8, comprising a component selected from the group consisting of a solvent, an acid generating agent, an acid crosslinking agent and a combination thereof.

10. A resist film formed from the composition according to claim 8.

11. A method for forming a pattern, comprising:
a film formation step of forming a film on a substrate by use of the composition according to claim 8,
an exposure step of exposing the film, and
a development step of developing the film exposed in the exposure step, to thereby form a pattern.

12. An underlayer film for lithography formed by use of the composition according to claim 8.

13. An optical component formed by use of the composition according to claim 8.

14. A tellurium-containing compound prepared by reacting
(a) a compound having one cyclic (thio) ether group, and
(B) TeX₄, wherein X is a halogen atom.

15. A composition comprising the tellurium-containing compound according to claim 14.

16. A purification method comprising:
a step of dissolving the tellurium-containing polymer according to claim 1 in a solvent comprising an organic solvent incompatible with water to thereby obtain a solution, and
a first extraction step of bringing the resulting solution into contact with an acidic aqueous solution to extract impurities in the polymer or compound.

17. A purification method comprising:
a step of dissolving the tellurium-containing compound according to claim 14 in a solvent comprising an organic solvent incompatible with water to thereby obtain a solution, and
a first extraction step of bringing the resulting solution into contact with an acidic aqueous solution to extract impurities in the polymer or compound.
